(19) United States
(12) Patent Application Publication
(10) Pub. No.: US 2015/0290148 A1
(43) Pub. Date: Oct. 15, 2015

(54) METHOD OF REDUCING BRAIN CELL DAMAGE, INFLAMMATION OR DEATH

(71) Applicant: THE UNIVERSITY OF MONTANA, Missoula, MT (US)

(72) Inventors: David J. Poulsen, Missoula, MT (US); Thomas Federick Rau, Stevensville, MT (US)

(21) Appl. No.: 14/752,279

(22) Filed: Jun. 26, 2015

Related U.S. Application Data
(63) Continuation of application No. 13/940,101, filed on Jul. 11, 2013.
Application No. 13/940,101 is a continuation of application No. 12/954,596, filed on Nov. 24, 2010, now abandoned.
Application No. 12/954,596 is a continuation-in-part of application No. 12/395,665, filed on Feb. 28, 2009.
Application No. 12/395,665 is a continuation-in-part of application No. 12/438,518, filed on Mar. 24, 2009.
(60) Provisional application No. 61/264,124, filed on Nov. 24, 2009.
(60) Provisional application No. 60/839,874, filed on Aug. 24, 2006.

(51) Int. Cl. A61K 31/137

(52) U.S. Cl. CPC A61K 31/137

(57) ABSTRACT

A method of reducing the occurrence of brain cell damage or death caused by transient cerebral hypoxia, ischemia, brain inflammation or a traumatic brain injury (TBI) event. The method typically comprises identifying a subject with transient cerebral hypoxia, ischemia, brain inflammation or a TBI, and within 24 hours of onset of the condition, administering to the subject a continuous intravenous infusion dose of methamphetamine in an amount sufficient to reduce the occurrence of brain cell damage or death caused by the condition. Preferably, the dose is increased in response to a delay in administration. The invention also relates to a method for modulating cytokine expression within the brain to treat such conditions.

### CROSS-REFERENCE TO RELATED APPLICATIONS

This application is a continuation of U.S. application Ser. No. 12/954,596, filed Nov. 24, 2010, which claims the benefit of U.S. Provisional Application No. 61/264,124, filed Nov. 24, 2009, and U.S. application Ser. No. 12/954,596 is a continuation-in-part of U.S. patent application Ser. No. 12/395,665, filed Feb. 28, 2009, which is a continuation-in-part of U.S. patent application Ser. No. 12/438,518 filed Feb. 23, 2009, which is the National Stage of International Application No. PCT/US2007/076034, filed Aug. 15, 2007, which claims the benefit of U.S. Provisional Application No. 60/839,974 filed Aug. 23, 2006. All of the above applications are hereby incorporated by reference, each in its entirety.

### STATEMENT REGARDING FEDERALLY SPONSORED RESEARCH OR DEVELOPMENT

Research relating to this invention may have been supported in part by the National Institutes of Health (NIH) under Research Grant Nos. 5R21NS058541 and R01AG031184-01. Therefore, the U.S. Government may have certain rights in this invention.

### FIELD OF THE INVENTION

The present invention is directed to methods of reducing the occurrence of brain cell damage or death as well as methods of modulating cytokine expression within the brain.

### BACKGROUND OF THE INVENTION

Strokes are the leading cause of disability among adults, with over 80% involving ischemic insult. To date, no preventative or neuroprotective therapy has proven to be efficacious in humans. Amphetamines are one of the most extensively studied and promising group of drugs used to facilitate stroke recovery after neuronal cell damage has occurred (see (Martinsson and Eksborg 2004)). In rats, a single dose of amphetamines (e.g., dexamphetamine) administered 24 hrs after sensorimotor cortex ablation promotes hemiplegic recovery (Feeney et al. 1982). This beneficial effect has been confirmed in a variety of different focal injury models and species (Sutton et al. 1989; Hovda and Fenney 1984; Hovda and Feeney 1985; Schmanke et al. 1996; Dietrich et al. 1990; Stroemer et al. 1998). In each of these studies ischemic injury was modeled by the permanent ligation/embolism of a vascular component, or cortical ablation.

A different type of ischemic injury involves the transient interruption and reperfusion of blood flow to the brain. The hippocampus is extremely sensitive to this type of ischemic insult. In humans and experimental rodent models, brief ischemic episodes can result in the selective and delayed death of neurons located in the hippocampus, especially the pyramidal cells of the CA1 sector (Kirino 1982). This type of lesion impairs performance on cognitive tasks that involve spatial memory (Zola-Morgan et al. 1986; Squire and Zola-Morgan 1991). Although amphetamine administration is associated with improved behavioral recovery in models of focal ischemia or cortical ablation, the prior art reported that treatment with amphetamines does not reduce infarct volume and thus, is not a preventative or neuronal protectant. The prior art also suggest that amphetamines facilitate behavioral recovery after cortical injury by influencing brain plasticity (Gold et al. 1984) as well as resolution of diaschisis ((Hovda et al. 1987; Sutton et al. 2000). The prior art, however, further teaches that amphetamines do not improve recovery following certain types of injury including lesions in the substantia nigra (Mintz and Tomer 1986). The prior art teaches that administration of amphetamines (e.g., methamphetamine; MAP) prior to focal ischemia actually increases the infarct volume in cortical and striatal regions (Wang et al. 2001).

Methamphetamine induces the extracellular accumulation of dopamine at nerve terminals by modulating the activity of dopamine transporters (DAT) and vesicular monoamine transporters (VMAT). Exposure to high and/or repetitive doses of methamphetamine results in excessive dopamine release within the synaptic cleft, leading to toxic levels of aldehydes and quinones. In addition, dopamine can increase the production of hydrogen peroxide and nitric oxide resulting in the generation of the reactive nitrogen species (RNS) peroxynitrite (Krasnova and Cadet, 2009). High doses of methamphetamine are also linked to excessive glutamate release in the striatum and hippocampus resulting in excitotoxicity (Nash and Yamamoto, 1993). Increased extracellular glutamate levels have also been linked to RNS production and activation of calcium-dependent proteases and cytoskeletal damage. High doses of methamphetamine also alter energy metabolism resulting in decreased succinate dehydrogenase activity (complex II of the electron transport chain) leading to mitochondrial dysfunction (Quinton and Yamamoto, 2006). Thus, the combination of reactive oxygen species (ROS), RNS, excitotoxicity, and mitochondrial dysfunction are linked to methamphetamine-induced loss of dopaminergic nerve terminals throughout the ventral tegmental area, subtantia nigra, hippocampus, prefrontal cortex and cortex (Hanson et al., 1998).

In contrast, it has been suggested that activation of the Dl dopamine receptor (D1R) can elicit a neuroprotective response (Lee et al., 2002). Lee reported that the D1R interacts directly with the NMDA receptor (NMDAR). Activation of D1R may reduce NMDAR Ca2+ currents in hippocampal neurons and decrease excitotoxicity in a phosphoinositol-3 kinase (PI3K) dependent manner (Lee et al., 2002). The D2 dopamine receptors (D2R) may modulate AMPA receptor activity through indirect interactions with the GluR2 subunit via the N-ethylmaleimide sensitive factor (NSF). Activation of D2R may lead to a reduction in AMPA receptors at the cell surface (Zou et al., 2005). This process also involves an increase in phosphoinositol-3-kinase (PI3K) activation. In addition, D2R activation has been shown to protect rat cortical neurons from glutamate excitotoxicity by activating anti-apoptotic signaling through AKT and up-regulation of Bc1-2 expression (Kihara et al., 2002).

A need still exists for an effective and safe treatment that reduces the occurrence of brain cell damage or death after the occurrence of a transient cerebral hypoxia and/or ischemia, as well as traumatic brain injury. In particular, altering the physiological environment of the brain presents challenges due to the limited permeability of the blood brain barrier. The presently disclosed methods provide a means to induce neuroprotection and reduce inflammation within the brain.

### SUMMARY OF THE INVENTION

The present invention is directed to a method of reducing the occurrence of brain cell damage or death in a subject. In a preferred embodiment, the invention is directed to a method of reducing the occurrence of brain cell damage or death caused by transient cerebral hypoxia/ischemia condition, brain inflammation condition or a traumatic brain injury (TBI) event.

In one embodiment, the method comprises identifying a subject with a transient cerebral hypoxic and/or ischemic condition, and within 24 hours of onset of the condition, administering to the subject a continuous intravenous infusion dose of methamphetamine in an amount sufficient to reduce the occurrence of brain cell damage or death caused by the condition. Preferably, a bolus dose of methamphetamine is administered to the subject in addition to the continuous intravenous infusion dose. The bolus dose is typically administered as soon as possible after the occurrence of the condition, preferably before or at the initiation of the continuous intravenous infusion dose.

Typically, the transient cerebral hypoxic and/or ischemic condition is caused by loss of blood, a heart attack, strangulation, surgery (e.g., cardiac surgery or neurosurgical procedures), a stroke, air-way blockage, ischemic optic neuropathy, low blood pressure, diagnostic or therapeutic endovascular procedures, ischemic optic neuropathy, neo-natal hypoxia, or air-way blockage. It is understood, that the method may be used to treat any condition that causes brain cell damage due to the lack of oxygen and/or glucose reaching the brain cells for a temporary period of time.

In another embodiment, the method comprises identifying a subject having a TBI event and, within 24 hours of the event, administering methamphetamine to the subject in an amount sufficient to reduce the occurrence of brain cell damage or death caused by the TBI event. Preferably, the step of administering the methamphetamine to the subject comprises administering a bolus dose of methamphetamine and a continuous intravenous infusion dose. Administration of a bolus dose prior to or at the initiation of the continuous intravenous infusion dose is preferred.

The TBI event is any event wherein a significant amount of physical force or torsion is applied to the upper torso, neck, or head of an individual, wherein the force is sufficient to cause brain cell damage or death. Preferably the TBI event is selected from the group consisting of: whiplash, a blast wave impact, or blunt force trauma of sufficient force to cause brain cell damage or death. In a preferred embodiment, the present invention is directed to a method treating a blunt closed head injury to reduce the occurrence of brain cell damage or death caused by the injury.

In yet another preferred embodiment, the method comprises identifying a subject having a condition associated with brain inflammation (e.g., encephalitis, cerebritis, encephalomyelitis, or meningitis caused by a bacterial or viral infection) and, within 24 hours of onset of the condition, administering methamphetamine to the subject in an amount sufficient to reduce inflammation of the brain and/or the occurrence of brain cell damage or death caused by the condition.

Advantageously, the amount of methamphetamine administered is typically sufficient to increase the expression of IL-10 and/or decrease the expression of IL-6.

In certain preferred embodiments above, the methamphetamine is administered within 24, 18, 16, 14, 12, 10, 8, 6, 4, or 2 hours of onset of the condition, preferably via intravenous infusion. Furthermore, it is preferable to administer the continuous intravenous infusion for at least 6, 12, or 18 hours; and more preferably for at least 24 to 48 hours.

### BRIEF DESCRIPTION OF THE DRAWINGS

FIG. 1 shows the dose response for methamphetamine (MAP) added immediately after 60 min of oxygen-glucose deprivation (OGD). Propidium iodide (PI) uptake in rat hippocampal slice cultures (RHSC) 48 hrs post OGD. MAP decreases neuronal death after OGD at concentrations ranging from 1 μM to 2 mM. At concentrations above 2 mM profound neurotoxicity was observed. **=p<0.01, OGD vs. groups; One way ANOVA, Dunnets Post-hoc. Each bar represents a minimum of 10 slices.

FIG. 2 shows propidium iodide uptake in RHSC 48 hrs post OGD Time course of MAP treatment occurring after 60 min of OGD. MAP was added at 2, 4, 8, 16, and 24 hours after OGD. All time points showed a significant reduction in neuronal death, however, the 24 hr. time point showed a significant increase in neuronal death when compared to the untreated non-OGD control. *=p<0.05, OGD vs. groups; †=p<0.05 UTD vs. groups, One way ANOVA, Dunnets Post-hoc. Each bar represents a minimum of 10 slices.

FIG. 3 shows a comparison of dopamine levels in acute vs. cultured slices Dopamine in acute hippocami compared to RHSC after 7 days in culture. Dopamine was measured by HPLC analysis in acute slices and normalized to protein content.

FIG. 4 shows homovanillic acid (HVA)/Dopamine in acute and cultured hippocampi Cultured hippocampal slices show active metabolism of dopamine after 7 days, indicating the presence of functional dopamine neurons.

FIG. 5A-5C show the neuroprotective effects of (A) serotonin, (B) norepinephrine, and (C) dopamine on rat organotypic hippocampal slice cultures exposed to 60 minutes of oxygen-glucose deprivation.

FIG. 6 shows propidium iodide uptake in RHSC at 24 hrs post-OGD Dopamine dose response after 60 min of OGD. Dopamine shows a significant neuroprotective effect in RHSC after OGD. **=p<0.01, OGD vs groups; †=p<0.05 UTD vs groups, One way ANOVA, Dunnets Post-hoc. Each bar represents a minimum of 5 slices.

FIG. 7 shows PI uptake in RHSC at 24 hrs post-OGD. Antagonism of D1/D5 receptors decreases the neuroprotective effect of MAP. Antagonists and MAP present immediately after 60 min. of OGD. SCH23390 at 20 μM; **=p<0.01, OGD vs. groups; †=p<0.05 D1/D5 ant MAP OGD vs. map OGD One way ANOVA, Tukey's Post-hoc. Each bar represents a minimum of 9 slices.

FIG. 8 shows PI uptake in RHSC 24 hrs post-OGD. Antagonism of the D2 receptor decreases the neuroprotective effect of MAP. Antagonists and MAP present immediately after 60 min. of OGD. *=p<0.05, **=p<0.01, OGD vs. groups; †=p<0.01, D2 ant+MAP+OGD vs MAP OGD One way ANOVA, Tukey's Posthoc. Each bar represents a minimum of 9 slices.

FIG. 9 shows a TUNEL staining in RHSC 24 hrs post-OGD. Low dose MAP after OGD decreases apoptosis in a dopamine dependent manner. Antagonists and MAP present immediately after 60 min. of OGD. Dopamine at 100 μM; *=p<0.05, **=p<0.01, OGD vs. groups; †=p<0.05, D2 ant+MAP+OGD vs MAP OGD. One way ANOVA, Tukey's Post-hoc. Each bar represents a minimum of 5 slices.

FIG. 10 shows TUNEL staining in RHSC 24 hrs post-OGD. Dopamine receptor antagonists decrease the anti-apoptotic effect of MAP after OGD. Antagonists and MAP present immediately after 60 min. of OGD. *=p<0.05, OGD vs. groups; †=p<0.05,MAP OGD vs Groups; ‡=p<0.05 D2 ant MAP OGD vs. D1 ant MAP OGD; One way ANOVA, Tukey's Post-hoc. Each bar represents a minimum of 4 slices.

FIG. 11 shows a western blot analysis of AKT and phospho AKT at 1 hrs post-OGD. The presence of a D1/D5 or a D2 receptor antagonist decreases the effect of MAP on AKT phosphorylation after OGD. The use of a PI3K inhibitor (LY29002) blocked the MAP mediated increase in AKT phosphorylation after OGD. Antagonists and MAP present immediately after 60 min. of OGD. Statistical analysis included one way ANOVA, Dunnet's post-hoc. Each bar represents a minimum of 8 slices. All data normalized to β-actin.

FIG. 12 shows mean (+SEM) distance traveled in a novel open field apparatus. Animals were tested 24 hrs following 5-min 2-VO (Isch) or sham surgery (Sham). Following surgery (1-2 min), gerbils received methamphetamine (5 mg) or saline vehicle (0 mg). Gerbils were placed in the center region and permitted to explore the novel environment for 5 minutes and distance data were collected using an automated tracking system. Ischemic gerbils without methamphetamine treatment were significantly more active compared to the no drug sham group. Ischemic and sham gerbils treated with the drug were not different and drug treatment failed to significantly alter activity levels relative to the control condition. *P<0.05 vs. Isch +drug condition.

FIG. 13 shows histological rating scores of hippocampal sections evaluated 21 days after ischemic insult (Isch) or sham control surgery (Sham). Gerbils were treated with methamphetamine (5 mg) or vehicle (0 mg) 1-2 minutes following surgery. Damage to the hippocampal CA1 region was evaluated using a 4 point rating scale. A score of 0 (4-5 compact layers of normal neuronal bodies), 1 (4-5 compact layers with presence of some altered neurons), 2 (spares neuronal bodies with “ghost spaces” and/or glial cells between them), 3 (complete absence or presence of only rare normal neuronal bodies with intense gliosis of the CA1 subfield) was assigned for each animal. Analysis revealed that treatment with methamphetamine significantly reduced damage to the hippocampal CA1 following ischemic insult.

FIG. 14 shows photomicrographs of hippocampal sections processed 21 days after ischemic insult or sham procedure followed by administration of methamphetamine (5 mg/kg) or vehicle. A 5-min 2-VO resulted in the selective loss of pyramidal neurons in the hippocampal CA1 region (Panels C, D). As expected, sham surgery (Panels A, B) did not result in any neuronal cell loss. Gerbils treated with methamphetamine 1-2 minutes following ischemic insult failed to exhibit any damage to the hippocampus (Panels E, F). Sections were stained with cresyl violet. Scale bars=200 μm (A, C, E) and 60 μm (B, D, F).

FIG. 15 shows that methamphetamine treatment decreases neurological impairment as measured by modified neurological severity score. MAP infusion at range of doses exerts a neuroprotective when administered immediately after the delivery of a 4 cm embolic clot. *=p<0.05, One way ANOVA, Tukey's post-hoc. Each bar represents an n of 8.

FIG. 16 shows infarct size measured by TTC staining at 7 days post embolic stroke. Methamphetamine decreases infarct size at 0.5 and 1.0 mg/kg/hr. Male Wistar rats were given a constant infusion of MAP (24 hrs) at 0.5 mg immediately after middle cerebral artery embolic occlusion. On day 7, coronal slices were made at 2.0 mm and stained with TTC. *=p<0.05; n=8.

FIG. 17 shows the Neurological Severity Score in adult male Wistar rats treated with methamphetamine 6 hrs after embolic stroke. Treatment with methamphetamine significantly decreased neurobehavioral deficits in rats exposed to embolic stroke. Methamphetamine at 1 mg/kg/hr for 24 hrs IV infusion. ***=p<0.001, One way ANOVA Tukey's post hoc; MAP day 7 vs. Groups; n=7 for MAP; n=8 for saline.

FIG. 18 shows infarct data in adult male Wistar rats showing the percentage of brain loss in the ipsilateral hemishpere after embolic stroke. Data collected indicates treatment with methamphetamine beginning 6 hours after embolic stroke significantly reduces infarct size. Methamphetamine at 1.0 mg/kg/hr for 24 hrs IV infusion. **=p<0.01, Two tailed t-test.

FIG. 19 shows representative TTC stained images showing infarct size (white areas represent infarcted/dead tissue). The brain slices on the top row belong to an animal treated with 1 mg/kg/hr MAP. The animal on the bottom row was treated with saline for 24 hours. All treatments began 6 hours post-stroke.

FIG. 20 shows the Neurological Severity Scores in adult male Wistar rats treated with methamphetamine 12 hrs after embolic stroke. Treatment with methamphetamine significantly decreased neurobehavioral deficits. Methamphetamine at 1 mg/kg/hr for 24 hrs IV infusion. *=p<0.05, One way ANOVA Tukey's post hoc; MAP day 7 vs. Groups n=4 for MAP; n=7 for saline.

FIG. 21 shows infarct data in adult male Wistar rats showing the percentage of brain loss in the ipsilateral hemispheres after embolic stroke. Data indicates treatment with methamphetamine beginning 12 hours after embolic stroke significantly reduces infarct size. However, data collected shows a significant increase in brain loss when comparing animals treated 12 hrs after stroke and animals treated 6 hrs after stroke. Methamphetamine at 1.0 mg/kg/hr for 24 hrs IV infusion. **=p<0.01, Two tailed t-test*=p<0.05, one tailed t-test.

FIG. 22 shows neurological severity scores for adult male Wistar rats treated with saline or methamphetamine 3 hours after TBI. Significant differences are observed 7, 14, 21 and 30 days after TBI. Day 7 assessment showed MA treatment reduced neurological impairment by 52% vs. 23% in saline treated animals.

FIG. 23 shows the effect of methamphetamine on placement dysfunction of forelimb (foot fault test) after TBI in adult male Wistar rats. Significant differences in the percent of forelimb misses were observed in the saline or MA treated animals.

FIG. 24 shows the effect of methamphetamine on learning and memory after TBI in adult male Wistar rats.

FIG. 25 shows the effect of methamphetamine on a probe trial after TBI in adult male Wistar rats.

FIG. 26 shows the pressure delivered to the dura in adult male Wistar rats suffering from a TBI.

FIG. 27 shows the righting reflex times in rats suffering from a TBI.

FIG. 28 shows the effect of methamphetamine on body weight after a TBI in adult male Wistar rats.

FIG. 29A-29D show the neuroprotective effect of methamphetamine following embolic focal ischemia. (A) Shows results of adhesive tape removal test in saline treated controls and animals infused with methamphetamine as indicated beginning at 0, 6 or 12 hours after stroke. Animals were tested on day 1 following stroke (black bars) and again 7 days after stroke (white bars). Values show the time required for animals to remove adhesive tape from both fore paws. Error bars represent mean±SEM. Each bar represents a minimum of 8 animals. (B) Shows results of infarct volumes measured at 7 days post stroke in animals treated with saline (black bars) or methamphetamine (white bars) immediately after stroke (top graph), beginning 6 hours post stroke (middle graph) or beginning 12 hours post stroke (bottom graph). Error bars represent mean±SEM. Each bar represents a minimum of 8 animals. (C) Shows neurological severity score (NSS) assessments on day 1 (black bars) and day 7 (white bars) following stroke for animals treated with saline or methamphetamine as indicated, immediately after stroke (top graph), beginning 6 hours post stroke (middle graph) or beginning 12 hours post stroke (bottom graph). Error bars represent mean±SEM. Each bar represents a minimum of 8 animals. (D) Shows foot fault assessments on day 1 (black bars) and day 7 (white bars) following stroke of animals treated with saline or methamphetamine as indicated immediately after stroke (top graph), beginning 6 hours post stroke (middle graph) or beginning 12 hours post stroke (bottom graph). Error bars represent mean±SEM. Each bar represents a minimum of 8 animals.

### DETAILED DESCRIPTION OF THE PREFERRED EMBODIMENTS

The present invention provides a method of reducing the occurrence of brain cell damage or death typically caused by transient cerebral hypoxia, ischemia, or a traumatic brain injury. The method comprises the steps of identifying a subject suffering from transient cerebral hypoxia, ischemia, or a traumatic brain injury, and within 36 hours of the onset of the condition or injury, administering to the subject a continuous intravenous infusion dose of methamphetamine in an amount sufficient to reduce the occurrence of brain cell damage or death caused by the condition. Preferably, administration begins within 24 hours after onset of the condition or injury, and more preferably within 16 hours. Still more preferably, administration begins within 6 to 12 hours after onset of the condition or injury, and most preferably in less than 6 hours.

Transient cerebral hypoxia and/or ischemia can be caused by many conditions that cause lack of oxygen and/or glucose to the cerebral cells for a temporary period of time. For example, a heart attack, strangulation, surgery (e.g., cardiac surgery), a stroke, blood loss, air-way blockage, or low blood pressure. The step of identifying a subject with transient cerebral hypoxia and/or ischemia can include identifying a subject having sudden numbness or weakness of the face, arm or leg, especially on one side of the body; sudden inability to talk or understand what is being said; sudden confusion or disorientation; sudden trouble seeing in one or both eyes; sudden trouble walking, dizziness, loss of balance or coordination; and sudden, server headache with no know cause. Preferably, the step further involves medical diagnostic techniques well known to those skilled in the art to further identify the specific condition, but use of such diagnostic techniques it is not required by the present invention.

In a preferred non-limiting example, the traumatic brain injury (TBI) is selected from the group consisting of: whiplash, a blast wave impact, or blunt force trauma of sufficient force to cause brain cell damage or death. The TBI can be identified by a chart or device showing impact forces for different impact events, e.g., blast, car collision at 30 miles an hour, etc. An example of a device for measuring impact force is a device worn by a soldier (e.g., helmet attachable) or part of a vehicle that can measure the pressure difference cause by a blast wave or blunt force impact, see for example U.S. patent application Ser. No. 12/154166, entitled “Soft tissue impact assessment device and system,” which incorporated by reference herein.

An event causing a traumatic brain injury is defined herein as any event in which a significant amount of physical force or torsion is applied to the upper torso, neck, or head of an individual, wherein the force is sufficient to cause brain cell damage or death. According to the invention, a TBI event does not require a loss of consciousness. Significant research into the field of traumatic brain injuries clearly demonstrates that a TBI event can cause brain cell damage or death, even without the subject sustaining a loss of consciousness. The TBI event can be any event in which the brain is subjected to a mechanical force that overcomes the opposing fluid force of cerebral spinal fluid, wherein the force is sufficient to induce brain cell damage or death. Non-limiting examples include a focalized, closed head physical contact, concussive blast wave energy, whiplash events (impulse events in which the head has suddenly, forcefully changed direction and velocity) and open wound brain damage in which the skull and dura are penetrated by a foreign object. A TBI event may further be defined as any event in which the individual's normal activity level (basal functioning) is interrupted by impact event.

A traumatic brain injury does not require a physical presentation of neurological symptoms in the subject. Advantageously, the methamphetamine can be administered after a TBI event even prior to the physical manifestation of neurological systems of brain cell damage or death. Slight to moderate TBI events have even been shown to induce neurological damage that may take months to manifest as physical symptoms. For example, a solider subject to concussive blast wave energy in the field is preferably immediately identified and administered a low dose of methamphetamine. Any individual that has been exposed to a significant amount of physical force or torsion applied to the upper torso, neck, or head area would preferably be administered methamphetamine in an amount sufficient to reduce the occurrence of brain cell damage or death.

Preferably, the method further comprises adjusting the dose of methamphetamine administered to the subject based on the amount of time between onset of the condition or injury and initial administration of methamphetamine. When administration begins less than six hours after the onset of the condition or injury, the dose is preferably greater than 0.1 mg/kg/hr and less than or equal to 1.0 mg/kg/hr. More preferably, the dose is greater than 0.1 mg/kg/hr and less than or equal to 0.5 mg/kg/hr. Conversely, when administration begins six hours or more following onset of the condition or injury, the dose is preferably greater than or equal to 0.5 mg/kg/hr and less than or equal to 1.5 mg/kg/hr. More preferably, the dose is greater than or equal to 0.5 mg/kg/hr and less than or equal to 1.0 mg/kg/hr.

The continuous intravenous infusion dose is preferably administered for at least 6 hours, more preferably for at least 12, 18, 24 or 48 hours. For example, the continuous intravenous infusion dose is typically administered for between 6 to 48 hours. The amount of methamphetamine used in the continuous intravenous infusion dose is preferably about 0.5 mg/kg/hr or less. When treating a human, the continuous dose is typically about 0.07 mg/kg/hr or less. For example, a preferred continuous dose is typically between about 0.001 mg/kg/hr and 0.05 mg/kg/hr.

Preferably the method further comprises administering a bolus dose of methamphetamine to the subject in addition to the continuous intravenous infusion dose. Typically, the bolus dose is administered as soon as possible after on set of the condition, e.g., within 18 hours, 16 hours, 12 hours, and most preferably within 6 hours. The amount of methamphetamine used in the bolus dose is typically not more than 0.5 mg/kg, especially in humans the bolus dose amount is typically not more than 0.18 mg/kg, for example, a preferred dose is about 0.12 mg/kg in humans.

In one embodiment, the amount of methamphetamine administered is sufficient to obtain a steady state plasma concentration of about 0.01 mg/L to about 0.3 mg/L in less than an hour, more preferably about 0.01 mg/L to about 0.05 mg/L.

It is preferable that the total amount of methamphetamine administered during a 24-hour period be 40 mg or less, especially when treating a human. This amount includes both the bolus dose amount and continuous dose amount administered during a 24 hour period.

The methods of the invention advantageously typically reduce the occurrence of brain cell damage in the hippocampus, striatum, or cortex of the brain.

In a specific embodiment of the invention, the method of reducing the occurrence of brain cell damage or death consists essentially of administering methamphetamine to the subject. In this specific embodiment, no other neurologically active ingredients beside methamphetamine are administered to the subject.

In another embodiment, the present invention relates to a method of inducing neuroprotection by modulating cytokine expression within the brain, the method comprising administering to a subject a dose of methamphetamine in an amount sufficient to modulate cytokine expression within the brain. As used herein, neuroprotection means a physiological state within the brain that diminishes the risk of brain cell damage or death from hypoxia, ischemia, traumatic brain injury, or inflammation. Thus, inducing neuroprotection is advantageous not only when a subject has suffered from hypoxia, ischemia, traumatic brain injury, or inflammation, but also when a subject is likely to suffer from such a condition or injury. For example, subjects going into surgery or suffering from a heart attack, soldiers in the field, or athletes playing contact sports may have a heightened need for inducing neuroprotection.

An amount sufficient to modulate cytokine expression within the brain may vary depending on the means of administration. Preferably, the dose of methamphetamine is administered via continuous intravenous infusion, and/or a bolus injection. If administered via a single intravenous bolus injection only, the amount of methamphetamine sufficient to modulate cytokine expression is preferably between 0.5 and 1.5 mg/kg, more preferably between 0.8 and 1.2 mg/kg, and most preferably 1.0 mg/kg. If administered via continuous intravenous infusion, the amount of methamphetamine sufficient to modulate cytokine expression is preferably greater than 0.1 mg/kg/hr and less than or equal to 1.5 mg/kg/hr, and most preferably between 0.5 mg/kg/hr and 1.0 mg/kg/hr. The continuous intravenous infusion dose is preferably administered for at least 6 hours, and more preferably between 6 and 48 hours. In humans, the amount of methamphetamine used in the bolus dose when administered in conjunction with continuous intravenous infusion is typically not more than 0.18 mg/kg, for example, a preferred dose is about 0.12 mg/kg in humans.

Furthermore, modulating cytokine expression comprises increasing expression of at least one cytokine and decreasing expression of at least one cytokine More preferably, modulating cytokine expression comprises increasing expression of IL-10 and decreasing expression of IL-6. Most preferably, modulating cytokine expression comprises increasing expression of IL-10 at least four-fold, and decreasing expression of IL-6 at least two-fold.

In another preferable embodiment, modulating cytokine expression comprises inducing an anti-inflammatory response within the brain. Preferably, increasing the expression of IL-10 within the brain induces an anti-inflammatory response.

The invention further relates to a method treating a subject having a condition associated with brain inflammation. Generally, brain inflammation refers to any condition causing swelling within the brain. Depending on the underlying cause and area inflamed, inflammation may be termed encephalitis, cerebritis, encephalomyelitis, or meningitis. The inflammation is usually caused by a reaction of the body's immune system to an infection or invasion by bacteria and viruses. Inflammation, however, may be caused by other micro-organisms (e.g., fungi and parasites) or by various non-infectious means. During the inflammation, the brain's tissues become swollen. The combination of the infection and the immune reaction can cause headache and a fever, as well as more severe symptoms in some cases. Inflammation may be caused by chickenpox, measles, mumps, Epstein-Barr virus (EBV), cytomegalovirus infection, HIV, herpes simplex, herpes zoster (shingles), herpes B, polio, rabies, mosquito-borne viruses (arboviruses; e.g., St. Louis encephalitis, California encephalitis, and Japanese encephalitis), Creutzfeldt-Jakob disease, lupus, Lyme disease, cancer, drugs (e.g., non-steroidal anti-inflammatory drugs, antibiotics and intravenous immunoglobulins), neurosarcoidosis, vasculitis, Behçet's disease, epidermoid cysts, dermoid cysts, and vaccinations.

Furthermore, increasing the expression of IL-10 contributes to neuroprotection by blocking apoptosis. Specifically, increasing the expression of IL-10 preferably increases expression of Bc1-2 and Bc1-x_{L}. Furthermore, increasing the expression of IL-10 preferably activates CREB and NF-kB, which in turn, increases expression of various neurotrophins. Preferably, these neurotrophins are selected from the group consisting of BDNF, NT3, and NPY. In yet another embodiment, the present invention relates to a method of inducing neuroprotection by activating monoamine receptors within the brain, the method comprising administering a compound via continuous intravenous infusion that is capable of rapidly crossing the blood brain barrier in an amount sufficient to induce the release of monoamines and inhibit the activity of monoamine transporters within the brain, further comprising modulating cytokine expression within the brain. As used herein, the blood brain barrier refers to a separation of circulating blood and cerebrospinal fluid (CSF) in the central nervous system (CNS). It occurs along all capillaries and consists of tight junctions around the capillaries that do not exist in normal circulation. Endothelial cells restrict the diffusion of microscopic objects (e.g. bacteria) and large or hydrophilic molecules into the CSF, while allowing the diffusion of small hydrophobic molecules (e.g., oxygen, hormones, and carbon dioxide). Cells of the barrier actively transport metabolic products such as glucose across the barrier with specific proteins. Particular examples of compounds that rapidly cross the blood brain barrier are known to those skilled in the art. As a specific non-limiting example, methamphetamine rapidly crosses the blood-brain barrier.

As used herein, monoamines refer to neurotransmitters and neuromodulators that contain one amino group that is connected to an aromatic ring by a two-carbon chain. Furthermore, monoamines are derived from aromatic amino acids like phenylalanine, tyrosine, tryptophan, and the thyroid hormones by the action of aromatic amino acid decarboxylase enzymes. Preferably, monoamines are selected from the group consisting of serotonin, dopamine, and norepinephrine. Likewise, the preferable monoamine receptors and monoamine transporters correspond to serotonin, dopamine, and norepinephrine. Using dopamine as a specific non-limiting example, the preferred monoamine transporter may be the dopamine transporter (DAT), and the preferred monoamine receptors may be the D1 and/or D2 receptors. As is known by those skilled in the art, these preferred transporters and receptors may differ depending on the monoamine (e.g., serotonin transporter may be, among others, SERT, and serotonin receptor may be, among others, the 5-HT receptor; norepinephrine transporter may be, among others, NET, and norepinephrine receptor may be, among others, adrenergic receptors).

Advantageously, the monoamine receptors are not over-stimulated. Thus, moderate activation of the monoamine receptors is preferred. As used herein, moderate activation of monoamine receptors means activation to a level that does not result in neurotoxicity. Preferably, the amount sufficient to induce the release of monoamines and inhibit the activity of monoamine transporters within the brain is greater than 0.1 mg/kg/hr and less than or equal to 1.5 mg/kg/hr, and most preferably between 0.5 mg/kg/hr and 1.0 mg/kg/hr.

### Compositions

Preferably the methamphetamine is in a pharmaceutical composition to be administered to the subject. The notation “methamphetamine” signifies the compounds of the invention described herein or salts thereof, including specifically the (+)-methamphetamine form. Pharmaceutical compositions and dosage forms of the invention typically comprise a pharmaceutically acceptable carrier.

In one embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which an active ingredient is administered. Such pharmaceutical carriers can be liquids, such as water and oils, including those of petroleum, animal, vegetable or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. In addition, other excipients can be used.

Preferably, the subject being treated by the methods is a mammal, e.g., monkey, dog, cat, horse, cow, sheep, pig, and more preferably the subject is human.

Unit dosage forms of the invention are preferably suitable for parenteral (e.g., subcutaneous, intravenous, bolus injection, intramuscular, or intraarterial), or transdermal administration to a patient. Examples include liquid dosage forms suitable for parenteral administration to a patient; and sterile solids (e.g., crystalline or amorphous solids) that can be reconstituted to provide liquid dosage forms suitable for parenteral administration to a patient. The methamphetamine is preferably administered via a bolus dose followed by a continuous intravenous dose, but other routes are contemplated.

Typical pharmaceutical compositions and dosage forms comprise one or more excipients. Suitable excipients are well known to those skilled in the art of pharmacy, and non-limiting examples of suitable excipients are provided herein. See, e.g., Remington's Pharmaceutical Sciences, 18th ed., Mack Publishing, Easton Pa. (1990).

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Frequency of dosage may also vary depending on the compound used and whether an extended release formulation is used. However, for treatment of most conditions or traumatic brain injuries, a bolus dose followed by a continuous intravenous single dose is preferred.

### Parenteral Dosage Forms

Parenteral dosage forms can be administered to patients by various routes including, but not limited to, subcutaneous, intravenous, bolus injection, intramuscular, and intraarterial. Preferably the parenteral dosage form is suitable for intravenous delivery. The parenteral dosage forms of the invention are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### EXAMPLES

The present invention will now be illustrated by the following examples. It is to be understood that the foregoing are for exemplary purposes only and are not intended to limit the scope of the invention. One skilled in the art can appreciate that modification may be made without departing from the spirit or scope of the present invention as set forth in the claims.

### Example 1

### In Vitro Hippocampal Slice Studies

### 1.1 Materials and Methods

### Hippocampal Slice Culture Preparation:

All experimental animal procedures were approved by the University Institutional Animal Care and Use Committee. Neonatal rats (Sprague-Dawley) at postnatal Day 7 (P7) were decapitated and the hippocampi dissected out under sterile conditions. 400 μm transverse hippocampal slices were prepared with a Mcllwain tissue chopper and cultured on Millicell permeable membranes (0.4 μM pore size) in six well plates for 6 days at 37° C. in 5% CO2. Slices were maintained in a primary plating media for two days (50% DMEM (+) glucose, 25% HBSS (+) glucose, 25% heat inactivated horse serum, 5 mg/mL D-glucose (Sigma), 1 mM Glutamax, 1.5% PenStrep/Fungizone (Gibco), and 5 mL of 50× B27 (Gibco) supplement plus anti-oxidants that was changed every 24 hr. Next, the slices were placed in serum-free neurobasal medium (10 mL Neurobasal-A, 200 μL of 50× B27 supplement, 100 μL of 100× Fungizone, and 100 μL of 100× Glutamax) and this media was changed every 48 hr.

### Oxygen Glucose Deprivation and Cell Death:

For oxygen-glucose deprivation (OGD) experiments, a glucose free balanced salt solution (BSS) (120 mM NaCl, 5 mM KCl, 1.25 mM NaH2PO4, 2 mM MgSO4, 2 mM CaCl2, 25 mM NaHCO3, 20 mM HEPES, 25 mM sucrose; pH 7.3) was bubbled for 1 hr with 5% CO2 95% N2 at 10 L/hr. Cultured slices were placed in pre-warmed BSS for 15 minutes to remove intracellular glucose and then washed three times and transferred into deoxygenated BSS and placed in a 37° C. chamber (Pro-Ox) with an oxygen feedback sensor that maintained gas levels at 0.1% O2, 5% CO2, 94.4% Nitrogen for 60 min. After OGD, the slices were immediately transferred back into prewarmed neurobasal media (containing B27 without anti-oxidants) under normal oxygen conditions. Slices treated with MAP in the dose response study were placed in normal media containing 1 μM-8 mM MAP immediately after OGD while time course studies added 100 μM MAP at predetermined intervals after OGD. Neuronal damage was determined by staining slices with propidium iodide (PI; Molecular Probes, Eugene, Oreg.) and quantifying the relative fluorescence intensity (excitation 540/emission 630). Dye was added to the media at a concentration of 2 μM (Noraberg, 1999), at least 12 hours prior to OGD. Images were taken of the hippocampal slices prior to OGD to establish baseline fluorescence. After OGD slices were placed in normal media containing 2 μM PI and imaged again at 48 hours post-OGD using fluorescence optics with an Olympus IMT-2 microscope and a Hamamatsu camera. The total fluorescent intensity in each slice was determined using Image Pro Plus software and all values were expressed as percent change from untreated OGD. (Version 6.2; MediaCybernetics, Silver Springs, Md.).

### Monoamine Comparison

Organotypic rat hippocampal slice cultures were exposed to OGD and treated with propidium as before. Immediately after OGD, slice cultures are placed in warmed media under normoxic conditions. A total of 8 slices were incubated with a dose range (10 nM, 100 nM, 100 μM and 1 mM) of serotonin, nor-epinephrine or dopamine alone. Cultures were incubated for 24 hrs under normal conditions then imaged for propidium iodide fluorescence. Relative fluorescence intensity was used to quantify neuronal death and damage.

### Determination of Apoptosis Using TUNEL Staining:

Apoptotic neuronal death was measured by nick labeled DNA utilizing the TUNEL (Promega) assay. Slices were fixed in 4% paraformaldehyde for 20 min at room temperature, rinsed in PBS three times and removed from Millicell inserts using a #5 paintbrush. After removal slices were placed on glass slides and processed according to the manufacturer's protocol. Images were captured at 506/529 ex/em and analyzed using ImagePro software. All values obtained were normalized to the untreated OGD mean and expressed as a percent change from this value.

### Western Blot Analysis:

Rat hippocampal slices were harvested from inserts and pooled (4) in 200 μl of SDS lysis buffer with 5% protease inhibitor cocktail (Sigma). Tissue was ground for 30 seconds, sonicated for 5 seconds on ice water, and centrifuged at 14,000g at 4° C. for 10 min. Protein content was determined by Bradford assay and 30-50 μg of protein was prepared with Lamelli sample buffer and loaded into Long Life 10 well gels (4-20%; NuSep and VWR). The gels were transferred to a PVDF membrane (Biorad Immun-Blot; 0.2 μM pore size) for 60 min at 100 volts on wet ice, blocked in 5% non-fat dry milk prepared in TBST for 1 hour, and incubated overnight on a Stovall roller at 4° with primary antibody (Cell Signaling; AKT 1:1000, pAKT 1:1000) in 5% non-fat milk. Blots were incubated with secondary antibody (1:20000 AKT; 1:2000 pAKT; Thermo Scientific donkey anti-rabbit) in 5% BSA for 1 hour and then washed 3 times for 5 minutes in TBST. Washed blots were then developed with an Amersham ECL Plus kit (GE) and exposed for 5 min (15 captures) on a Bio Rad Chemidoc system. Densitometry was performed using Quantity One software. Blots were stripped using Restore Western Blot Stripping buffer (Pierce), washed three times in TBST, and blocked for 1 hour in 5% non-fat dry milk and TBST. Blots were incubated overnight at 4° with a monoclonal antibody for β-actin (Sigma) at 1:45,000 and developed with an Amersham ECL Plus kit (GE). All samples were normalized to β-actin values as a loading control prior to statistical analysis.

### 1.2 Results

### Low Dose MAP Decreases Neuronal Death in RHSC Exposed to OGD:

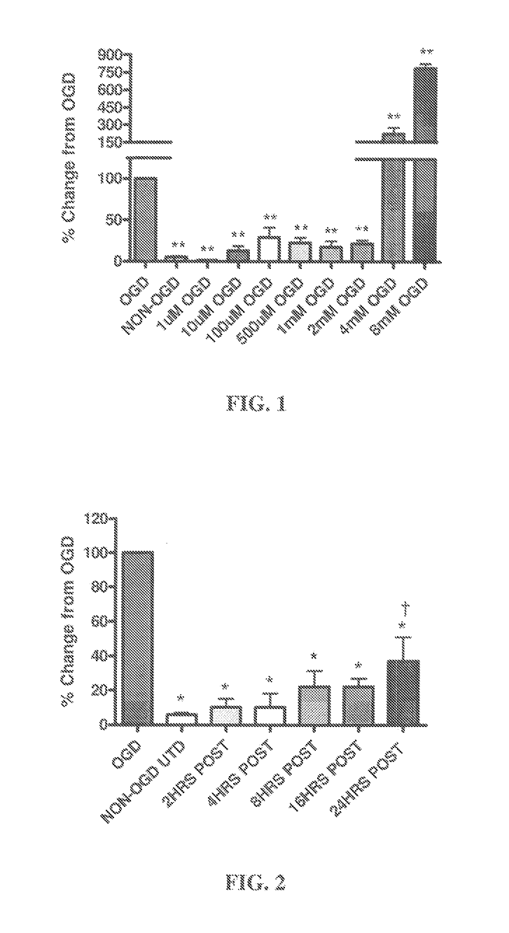

To examine the effect of MAP following OGD, rat hippocampal slice cultures (RHSC) were exposed to 60 min. of OGD and treated with MAP (1 μM-8 mM) immediately after the insult. Neuronal death was determined by staining cultures with propidium iodide (PI), and measuring the relative fluorescent intensity 24 hrs after stroke (Noraberg et al., 1999). MAP treatment after stroke resulted in a significant decrease in PI uptake over a broad dose range (1 μM-2 mM) when compared to untreated slices exposed to OGD (FIG. 1). The administration of higher doses of MAP (4 mM and 8 mM) resulted in a significant increase in neuronal damage following OGD. To investigate the time-dependence of MAP-mediated neuronal protection following OGD, 100 μM MAP was added at set points following 60 min of OGD. Analysis of PI uptake showed a significant decrease in neuronal death could be obtained when MAP was added up to 24 hrs following the initial insult (FIG. 2). Based on data collected in the RHSC model, it was shown that low dose MAP decreased cell death when added up to 24 hours after OGD. It appears that this protection may be occurring by inducing the release of dopamine and activating a neuroprotective mechanism through G-protein coupled dopamine receptors. Applicant also found that MAP induces the release and blocks the re-uptake of dopamine, and low dose dopamine has been shown to be neuroprotective through activation of G-protein coupled dopamine receptors. Hippocampal tissue was assayed to determine the quantity of dopamine present and whether it was in sufficient quantities to exert a significant neuroprotective effect.

### Rat Hippocampal Slice Cultures Contain Significant Amounts of Dopamine After 8 Days in Culture:

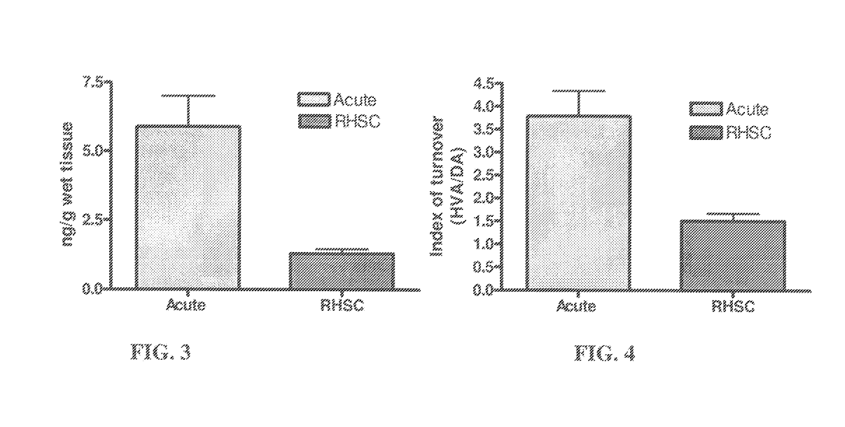

High performance liquid chromatography (HPLC) analysis of RHSC tissue showed hippocampal tissue contained a significant amount of dopamine after 8 days in culture (FIG. 3). Further analysis of RHSC tissue detected the presence of the dopamine metabolite, homovanilic acid (HVA) indicating dopamine was present, and dopaminergic neurons were actively metabolizing dopamine to HVA (FIG. 4). Analysis of acute slices showed a significantly higher percentage of dopamine and HVA suggesting dopamine from projection neurons originating in the ventral tegmental area (VTA) and the substantia nigra are directly contributing to dopamine signaling in the hippocampus. Analysis of cultured RHSC clearly demonstrated hippocampal tissue contains dopamine neurons irrespective of the input from projection neurons. To further test were conducted to test the efficacy of MAP at preventing neuronal death by inducing dopamine release. These experiments were conducted to test and further understand the effect of graded doses of dopamine after OGD.

### Mechanisms of Methamphetamine-Mediated Neuroprotection

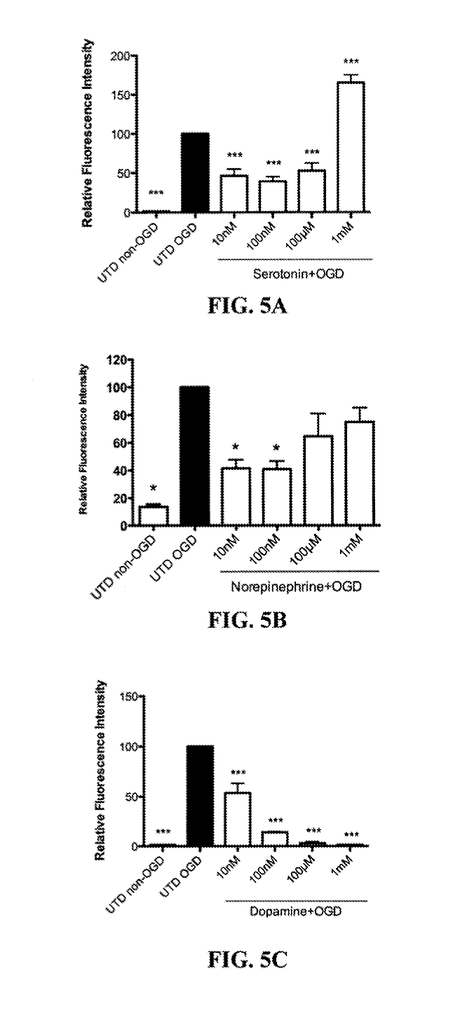

In addition to dopamine, methamphetamine induces the release of serotonin and norepinephrine. Therefore, we compared the ability of all three catecholamines, over a broad dose range (10 nM-1 mM), to induce a neuroprotective response in the RHSC-OGD model. Neuroprotection was observed with each of the monoamines individually. Serotonin produced a moderate neuroprotective response at doses of 10 nM-100 μM (FIG. 5a). However, the highest dose tested (1 mM) increased neuronal loss compared to the untreated OGD control. Norepinephrine also produced a moderate neuroprotective response but over a slightly smaller dose range (10 nM-100 nM) (FIG. 5b). In contrast, dopamine induced a potent dose-dependent neuroprotective response at all concentrations tested (FIG. 5c). PI staining of neurons in cultures exposed to OGD and treated with 100 nM-1 mM dopamine was similar to that seen in RHSC not exposed to OGD.

### Exogenous Dopamine Exerts a Neuroprotective Effect After OGD:

RHSC exposed to OGD and treated with graded doses dopamine after OGD showed a dose dependent decrease in neuronal death. From 10 nM up to 1 mM dopamine significantly reduced PI uptake when compared to untreated RHSC exposed to OGD (FIG. 6). While the 10 nM dose was significantly different from the untreated non-OGD control, the 100 nM-1 mM did not differ from the untreated, non-OGD control. This finding suggests dopamine, in sufficient quantities, is capable of exerting a significant neuroprotective effect in the hippocampus after OGD. To confirm this role of dopamine in MAP mediated neuroprotection, experiments with MAP were repeated in the presence of a D1/5R or D2R antagonist.

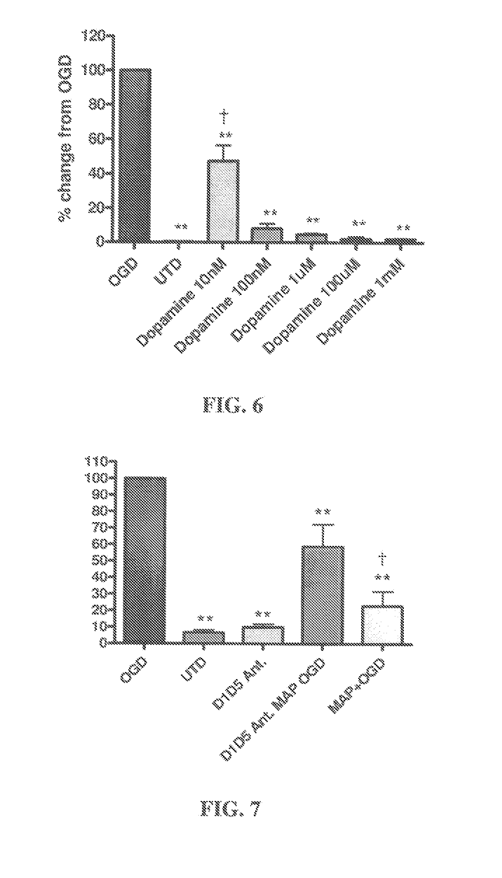

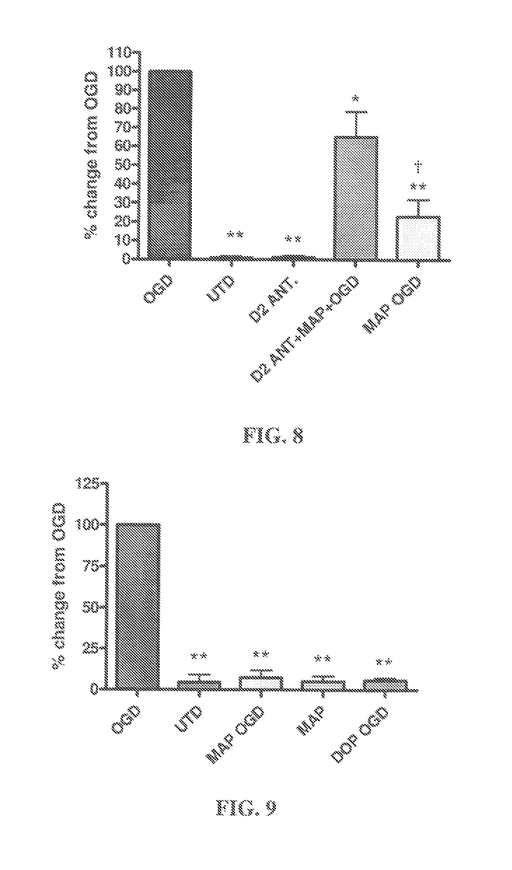

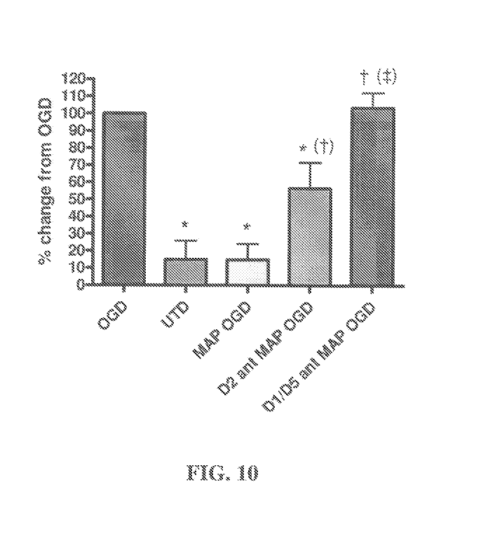

The administration of a D1/5R or D2R antagonist decreases the neuroprotective effect of MAP after OGD: RHSC were exposed to OGD, treated with the D1/5R antagonist SCH23390 or D2R antagonist raclopride, and treated with 100 μM MAP. The application of the D1/5R antagonist or the D2R antagonist significantly decreased the neuroprotective effect of MAP after OGD (FIGS. 7-8). This observation indicates MAP is exerting a neuroprotective effect in the hippocampus by modulating dopamine release and subsequent activation of both the D1/5R and the D2R. This observation is further supported by data showing antagonism of D1/5R receptor in the absence of MAP does not significantly differ from the untreated OGD group.

While PI uptake represents an effective tool for measuring neuronal death, it does not differentiate between necrosis and apoptosis. Having observed a significant decrease in neuronal death with MAP treatment, experiments were conducted to measure the effect of MAP on apoptosis after OGD using TUNEL staining to label apoptotic neurons.

### Low Dose MAP Decreases Apoptosis in Neurons Exposed to OGD and the Effect is Reduced by Dopamine Receptor Antagonists:

Untreated RHSC exposed to 60 min of OGD displayed widespread TUNEL staining throughout the CA1, CA2, CA3, and dentate gyms. RHSC treated with 100 μM MAP had a significant decrease in TUNEL positive neurons at 24 hrs post-OGD when compared to untreated OGD cultures (FIG. 9). This effect was measurably decreased when a D1/5R or D2R antagonist was added after OGD but prior to MAP treatment. However, antagonism of either receptor failed to completely abolish the neuroprotective effect of MAP (FIG. 10). These data suggest low dose MAP is reducing apoptosis after OGD in a dopamine dependent manner, and the reduction in apoptosis is not solely dependent on singular activation of the D1/5R or D2R. Downstream of the D1/5R and D2R is PI3K which in turn phosphorylates and activates the anti-apoptotic protein kinase AKT. To determine if PI3K was playing a role in MAP mediated decreases in apoptosis,

RHSC were treated with the PI3K inhibitor, LY29002. Results from this experiment show inhibition of PI3K disrupts the anti-apoptotic effect of MAP suggesting the neuroprotective effect of MAP has a key component in the PI3K-AKT signaling pathway.

### Low Dose MAP Increases Phosphorylation of PI3K and AKT (Protein Kinase B) in a Dopamine Dependent Manner:

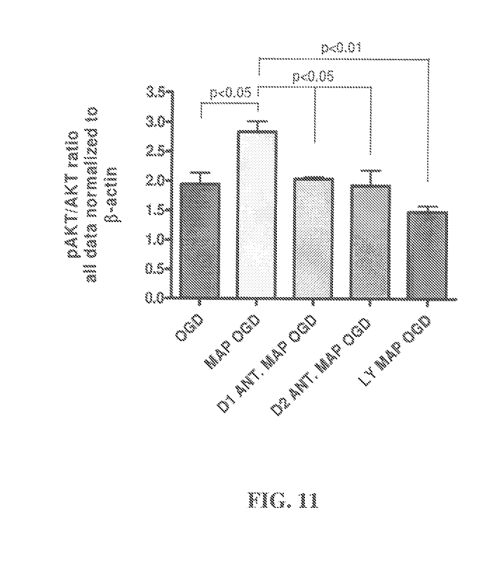

Western blot analysis of RHSC at 1 hour post-OGD showed MAP treatment increased the ratio of phosphorylated AKT to AKT, indicating MAP increases the kinase activity of AKT protein (FIG. 11). When MAP was added in the presence of the PI3K inhibitor, LY29002, AKT phosphorylation was significantly decreased suggesting the observed increase in AKT phosphorylation by MAP treatment is dependent on PI3K signaling. To determine if this effect was due to activation of dopamine receptors, western blot analysis was performed on samples treated with D1/5R or D2R antagonist and low dose MAP after OGD. Dopamine antagonists significantly decreased the MAP induced phosphorylation of AKT at 1 hours post-OGD, suggesting MAP mediated dopamine release is responsible for the increase in PI3K signaling and the subsequent increase AKT kinase activity.

### 1.3 Discussion

In the present study, experiments were conducted to test the hypothesis that low dose MAP would decrease neuronal death in hippocampal brain slices after acute oxygen glucose deprivation (OGD). The hippocampus is particularly susceptible to neuronal damage and death after oxygen glucose deprivation, and previous studies have shown relatively mild insults will produce regions of neuronal death within the hippocampus that do not appear in other areas of the brain (cortex, pre-frontal cortex) due to a high population of glutamatergic neurons that produce excitotoxic damage. A large number of hypoxia-ischemia studies have focused on excitatory amino acids (EAA) within the hippocampus, but relatively few studies have been conducted on the effects of catecholamine release and their subsequent activation of receptor groups within the hippocampus after OGD.

While neuroanatomical studies have clearly demonstrated the presence of dopamine projection neurons from the VTA and substantia nigra into the hippocampus, present data collected from HPLC analysis of isolated, cultured hippocampal slices clearly demonstrates the presence of both dopamine and the dopamine metabolite homovanilic acid (HVA). This finding indicates cultured hippocampal slices have a significant number of functional, metabolically active dopamine neurons. However, based on the amount of dopamine detected in cultured slices and the broad dosing range of MAP (1 μM to 2 mM) used to induce neuroprotection it appears the effect is limited to a relatively small amount of dopamine released within the isolated hippocampus.

Increasing the MAP dose up to 2 mM did not increase the neuroprotective effect, nor did it increase cell death; only at concentrations greater than 2 mM did cell death increase significantly. This observation suggests MAP at very low concentrations in the hippocampus may be suitable to induce the release of dopamine stores and exert a neuroprotective effect. This finding also suggests the cell death observed at 4 mM may not be due to dopamine toxicity as there are insufficient stores available to induce to ROS mediated neurotoxicity. In light of this finding, the specific mechanism responsible for neuronal death at high concentrations of MAP remains undefined. This observation is further supported by data collected from dopamine dose response experiments which showed a broad range of dopamine (10 nM-1 mM) exerted a neuroprotective effect and failed to induce toxicity (up to 1 mM). This finding suggests the limited amount of dopamine neurons present may be incapable of generating sufficient ROS, dopamine aldehydes, and quinones that have been implicated in dopamine-mediated neuronal death.

Previous studies of OGD in RHSC have shown an early necrotic form of cell death followed by a wave of apopototic death that begins at 6-8 hours post-OGD and continues up to 48 hours after the insult. In light of the time course data obtained (MAP was neuroprotective when added up to 24 hours post OGD; FIG. 2) it is likely that MAP is affecting mechanisms that modulate apoptotic death. This hypothesis was confirmed by TUNEL staining that demonstrated MAP treatment after OGD significantly decreased the number of apoptotic cells 24 hours after OGD. Based on this finding, the fact that MAP induces the release of dopamine, and the previous studies demonstrating activation of dopamine receptors decreases apoptosis, it was hypothesized the anti-apoptotic effect of MAP, at least in part, was mediated through dopamine receptors.

Antagonism of the D1/5R significantly decreased the neuroprotective effect of

MAP and resulted in a significant increase in apoptotic death when compared to the MAP treatment. Similarly, antagonism of the D2R receptors decreased the neuroprotective effect of MAP and resulted in a significant increase in neuronal death when compared to the untreated control. However, antagonism of the D1/5R completely blocked the antiapoptotic effect of MAP. In contrast, antagonism of the D2R decreased MAP-mediated neuroprotection from apoptosis, but slices had significantly less apoptotic cells when compared to the OGD only group (FIG. 9). This observation suggests MAP-mediated decreases apoptosis are more heavily dependent on activation of the D1/5R, and this observation may be explained by differential populations of dopamine receptors within the hippocampus. If this hypothesis is correct specific regions of the brain may show more differential anti-apoptotic effects based on receptor populations.

In an effort to study the downstream effects of MAP after OGD, western blots were performed on RHSC treated with MAP after OGD. Blots probed with AKT and phosphorylated-AKT showed MAP treatment after OGD significantly increased the percentage of active (phosphorylated; pAKT) AKT Inhibition of PI3K blocked the MAP-mediated increase in pAKT indicating the increase was dependent, at least in part, to PI3K activation. Further studies showed antagonism of both the D1/5R and D2R blocked MAP mediated increases in phosphorylated AKT. These findings suggest MAP treatment after OGD decreases apoptosis by activation of AKT through a PI3K-dopamine dependent mechanism.

AKT (Protein kinase B) is a critical, pro-survival kinase that has been shown to suppress a number of apoptotic mechanisms leading to neuronal protection after an insult. Previous studies involving hypoxia-ischemia have shown AKT suppresses activation of mitochondrially mediated cleaved caspase 9 in neurons. Further studies have determined AKT phosphorylation inactivates pro-apoptotic BAD by phosphorylating BAD binding protein, 14-3-3. The binding of 14-3-3 to BAD blocks the formation of the BAD-Bc1-xl complex and allows Bc1-xl to promote cell survival. AKT also stimulates activation of inhibitors of apoptosis, particularly XIAP, resulting in decreased initiation of apoptosis.

AKT, while effectively blocking apoptosis in neurons, also serves to promote cell survival by modulating the forkhead transcription factor FoxO1 and tumor suppressor p53. Previous studies have shown AKT directly phosphorylates FoxO1 at Thr24, Ser256 and Ser319, which results in nuclear export and inhibition of transcription factor activity leading to cell survival. To modulate p53 activity, AKT phosphorylates MDM2 which then binds to p53 and inhibits p53 accumulation by targeting it for ubiquitination and proteasomal degradation.

AKT has also been shown to modulate excitatory synaptic transmission, a key component of OGD-mediated damage. In studies performed by Wang et al, AKT was shown to phosphorylate the GABAA receptor on the β2 subunit at serine 410. The phosphorylation of GABAA by AKT significantly increased post-synaptic density of GABAA receptors resulting in a significant inhibition of excitatory amino acid signaling. In light of the observed decrease in neuronal death and apoptosis and the increase in AKT phosphorylation, it is possible low dose MAP treatment is targeting multiple cell survival mechanisms. Blocking apoptosis, promoting cell survival and decreasing excitatory synaptic transmission may be separate, distinct mechanisms that provide the downstream effectors responsible for the neuroprotection observed with low dose MAP after OGD.

Data collected from this study also suggests the involvement of other mechanisms unrelated to dopamine activation of PI3K. MAP experiments conducted in the presence of either a D1/5R or D2R type antagonist significantly decreased the neuroprotective effect of MAP but RHSC still showed a significant decrease in neuronal death when compared to the OGD group (FIGS. 6-7). Further supporting this hypothesis was experimental data showing the addition of both a D1/5R and D2R type antagonist failed to show an additive effect (data not shown) suggesting the neuroprotective mechanism(s) is not limited to activation of D1/5R and D2R receptor types. In light of the multiple effects of MAP on the release of norepinephrine, serotonin, and the upregulation of CART peptide, it appears likely MAP-mediated release of dopamine is not the sole mechanism responsible for the observed neuroprotective effect.

### Example 2

### Alterations in Gene Expression

### 2.1 Materials and Methods

Six adult, male Sprague-Dawley rats were injected IP with a single dose of 1 mg/kg methamphetamine. Four additional control rats received IP injections with equal volumes of saline. Three rats from each group were euthanized at one hour after injection. The remaining three were euthanized at six hours after injection. Both hippocampi from each animal were recovered and processed as separate samples. Changes in the expression of specific genes were determined by quantitative real time PCR analysis using the neurotrophin and receptor array and RT² Profiler PCR Array System according to the manufacturers instructions (SA Biosciences, Fredricksberg, Md.). Total RNA was isolated from hippocampal tissue then RNA quality and quantity was established with an Agilent 2100 bioanalyzer. Samples from each hippocampus were run in triplicate and analyzed using software provided by SA bioscience. Only genes that showed a statistically significant change (p<0.05) were considered valid targets.

### 2.2 Results

To further elucidate the potential mechanisms of methamphetamine-mediated neuroprotection, we used quantitative real time PCR analysis to compare the expression levels of selected genes within the hippocampus of methamphetamine-treated rats. Methamphetamine has a half-life of approximately 1 hr in rats¹. Therefore, we examined gene expression changes at 1 and 6 hours after single intraperitoneal injection of either methamphetamine (1.0 mg/kg) or saline.

At 1 hour after methamphetamine injection, expression of the interleukin 10 (IL10) gene increased by 445%. The increased expression of IL10 was associated with a significant decrease in the expression of the interleukin 6 (IL6) gene (−203%). This suggests that, at this dose, methamphetamine establishes a strong anti-inflammatory condition shortly after treatment. At 6 hours following methamphetamine administration, IL10 and IL6 expression were not significantly different from saline treated animals. However, at 6 hours after treatment, gene expression for BcL2 (+167%), brain derived neurotrophic factor (+141%), neuropeptide Y (+192%) and neurotrophic factor 3 (+192%) were all increased in the hippocampus of methamphetamine treated animals when compared to saline injected controls.

### 2.3 Discussion

Methamphetamine may induce neuroprotection via increased expression of IL10, which in turn inhibits the expression of the pro-inflammatory cytokine IL6. We showed that IL10 expression increased more than four fold within 1 hour after a single injection of 1.0 mg/kg of methamphetamine. The increase in IL10 coincided with a greater than two fold reduction in IL6 expression. In addition to inducing an anti-inflammatory response, the methamphetamine-mediated increase in IL10 may also contribute to neuroprotection by blocking apoptosis. IL10 increases expression of Bc1-2 and Bc1-x_{L}. Bc1-2 expression increased following treatment with methamphetamine, which was delayed in relation to the increase in IL10 expression. We also saw a delayed increase in BDNF, NT3 and NPY expression, which followed the increase in IL10. Expression of these neurotrophins is modulated by NF-kB and CREB. The pro-survival effects of IL10 in neurons are mediated in part by AKT signaling and activation of CREB. In addition, IL10 induces gene expression changes in neurons via activation NF-kB.

In sum, treatment with methamphetamine elicits changes in gene expression that prevent apoptosis by increasing Bc1-2 expression, and promotes neuronal survival by increasing expression of BDNF, NPY and NT3. In addition, methamphetamine may lead to an anti-inflammatory response through reduction of IL-6 and increased expression of IL-10.

### Example 3

### In Vivo Transient Cerebral Ischemia

### 3.1 Materials and Methods

### Induction of Transient Cerebral Ischemia:

Gerbils were anesthetized with isoflurane and core-body temperature maintained at 37-38 C during surgery using a homeothermic blanket (Harvard Apparatus, South Natick, USA). A midline incision was made in the neck and the common carotid arteries were isolated and occluded for 5 min using 85-gm pressure aneurysm clips (ISCH; n=14). A second group of gerbils (SHAM; n=14) underwent the identical procedure except the carotid arteries were not clamped. The incision was sutured and animals received MAP (5 mg/kg; i.p) or equal volume of vehicle (saline; 0 mg) within 2 minutes of reperfusion. Animals were placed in a warmed cage, and observed for 30 minutes. Tylenol (8 mg/ml) was added to drinking water to provide postoperative analgesia.

### Behavioral Testing and Histological Evaluation:

Each gerbil was tested 48 hrs following surgery in an open-field apparatus consisting of a metal screen floor 77 cm×77 cm with walls 15 cm in height. Animals were placed in the center region and permitted to explore the novel environment for 5 minutes. Behavioral data (distance traveled, speed) were collected using an automated tracking system (ANY-maze, Stoelting, Ill.) and evaluated separately using ANOVA and the appropriate post hoc test (P<0.05 considered significant). Twenty-one days postsurgery, gerbils were euthanized with CO2 and perfused with phosphate buffered saline followed by 4% paraformaldehyde. Tissue from sham gerbils treated with MAP (SHAM+0 mg) was not evaluated since acute administration of MAP was not expected to histologically alter the hippocampus of this group. Brains were removed and post-fixed for at least 48 hrs prior to collection of 40 μm vibratome sections through the hippocampal region. Sections were mounted on slides and stained with cresyl violet. Damage to the hippocampal CA1 region was evaluated without knowledge of treatment condition by two independent observers using a 4 point rating scale described elsewhere (Babcock et al. 1993). A score of 0 (4-5 compact layers of normal neuronal bodies), 1 (4-5 compact layers with presence of some altered neurons), 2 (spares neuronal bodies with “ghost spaces” and/or glial cells between them), 3 (complete absence or presence of only rare normal neuronal bodies with intense gliosis of the CA1 subfield) was assigned for each animal. Ratings were averaged and evaluated using nonparametric statistics (Kruskal-Wallis and Mann-Whitney U test; P<0.05 considered significant).

### 3.2 Results

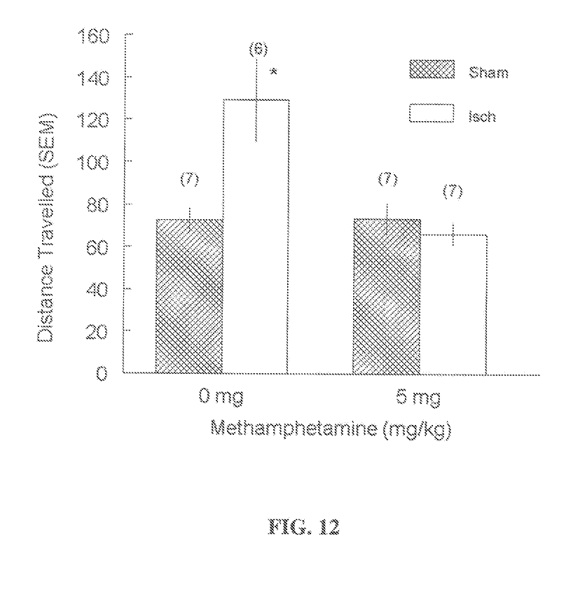

Gerbils exhibited coordinated movements within 10 minutes of isoflorane termination. Animals treated with MAP became piloerect with their tails pointing up. Animals were tested in an open field apparatus 48 hrs following surgery. Gerbils that underwent ischemic insult without MAP treatment traveled 129.4 m (±20; SEM), while sham controls with and without drug treatment traveled 72.7 m (±6) and 73.2 m (±7.5), respectively (FIG. 12). Ischemic gerbils treated with MAP following surgery traveled 66.3 m±5.6. Analysis of activity data revealed a significant interaction between drug treatment and surgical conditions (P<0.05). Subsequent planned comparisons indicated that ischemic gerbils, in the absence of MAP treatment, were significantly more active compared to the no drug sham group (P<0.05). Ischemic and sham gerbils treated with MAP were not significantly different (P>0.05). Finally, treatment with MAP failed to significantly alter activity levels relative to the control condition (SHAM+0 mg vs. SHAM+5 mg; P>0.05). Analysis of speed data (distance traveled/time) revealed a similar pattern with ischemic gerbils treated with saline (ISCH exhibiting significantly fastest speeds relative to all other experimental groups (data not shown).

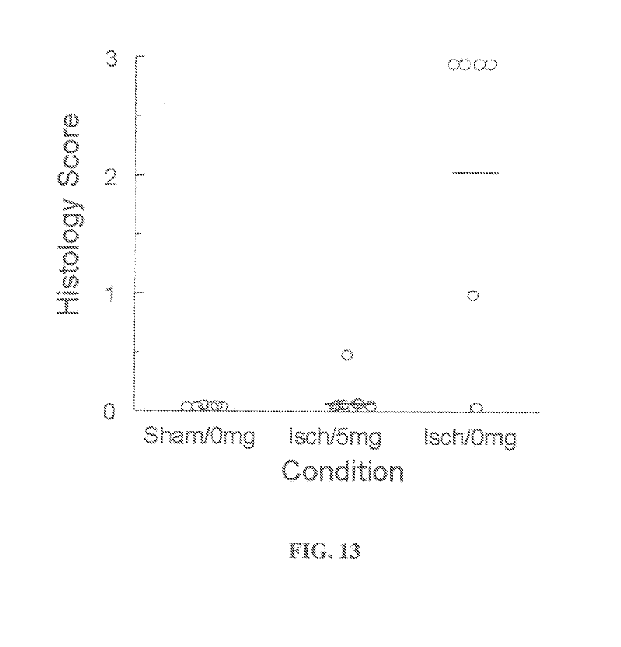

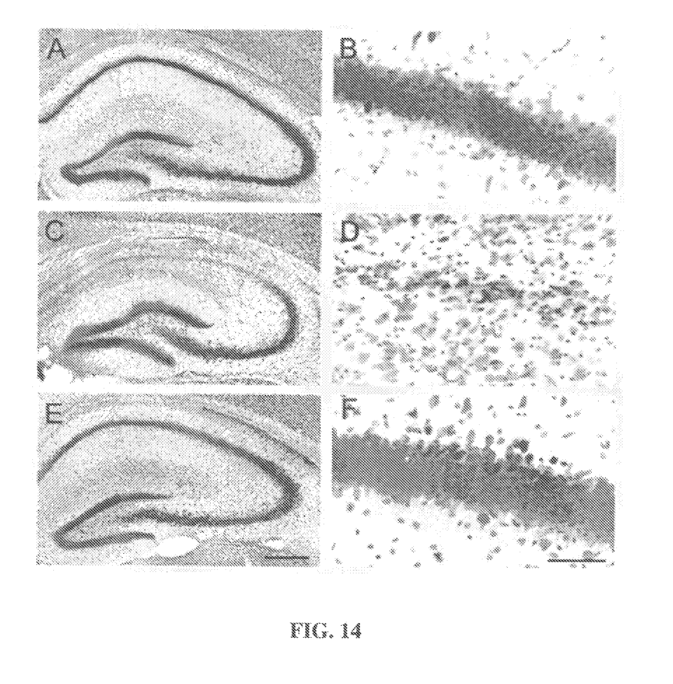

The histopathology scores and representative photomicrographs of the evaluated groups are illustrated in FIGS. 13-14, respectively. Gerbils in the ISCH+0 mg condition exhibited extensive damage to the hippocampal CA1 region. Four of six gerbils in this group had complete absence of normal neuronal bodies with intense gliosis of the CA1 subfield. In contrast, all of the gerbils in the SHAM+0 mg group were rated as having no detectable damage to the hippocampus (mean rating 0±0). Six of the animals in the ISCH+5 mg MAP group exhibited 4-5 compact layers of normal neuronal bodies in the hippocampus (group rating 0.07±0.07). Only 1 gerbil in this condition exhibited any detectable damage to the CA1 region. Analysis of rating scores revealed a significant difference between groups (P<0.05). Subsequent evaluation of individual group data indicated that SHAM+0 mg and ISCH+5 mg conditions were not significantly different (P>0.05) and both of these conditions were significantly different from the ISCH+0 mg group (P<0.05).

### 3.3 Discussion

The neuroprotective efficacy of MAP was demonstrated in vivo using a 5-min gerbil 2-VO transient ischemia model. MAP administration within 1-2 minutes of reperfusion prevented any significant loss of hippocampal CA1 pyramidal cells. The histological evaluation revealed that ischemic gerbils treated with MAP exhibiting almost complete protection of the hippocampal CA1 region with only 1 of 7 animals exhibited any detectable neuronal pathology in the hippocampus. A 5-min bilateral carotid occlusion in the gerbil produces increased locomotor activity that correlates with hippocampal CA1 cell death (Wang and Corbett 1990; Babcock et al. 1993). The locomotor activity of ischemic gerbils treated with MAP in the present study was comparable to control levels, which is indicative of significant neuroprotection. It is entirely possible that the arousal and hyperactivity that amphetamines produce could interact with the behavioral effects of ischemia. However, behavioral testing in the present study was conducted after the drug should have been metabolized (48 hrs). Consistent with this interpretation was the observation that control gerbils treated with MAP were not hyperactive relative to animals that received saline (SHAM+0 mg). The dose of MAP used in the in vivo experiment was derived from a previous report that used gerbils (Teuchert-Noodt et al. 2000; Araki et al. 2002) as an experimental model. We also conducted a preliminary study in which doses of MAP greater than 5 mg/kg (e.g., 10 and 20 mg/kg) were found to be lethal in gerbils following surgery and were not evaluated further.

Amphetamine administration in combination with training has been shown to be a promising pharmacological strategy for behavioral recovery after stroke (see Martinsson and Eksborg, 2004). It is notable that these findings show that neuroprotection is independent of any behavioral training following the insult. Unlike focal ischemia or other types of cortical injury, transient cerebral ischemia is characterized by a pattern of delayed cell death limited to hippocampal pyramidal cells. The reperfusion that follows the brief ischemic episode in this model is a key event for the subsequent cell death that occurs 3-5 days following insult.

Current studies of MAP administration prior to an acute stroke event indicate that MAP significantly increases neuronal death (Wang et al. 2001). However, in light of our current findings, it is entirely possible that treatment with MAP prior to a stroke event depletes stores of dopamine and norepinephrine that remain unavailable for release after a stroke event, and the subsequent decrease in neuronal signaling may be playing a key role in the damage observed in MAP pre-treatment and stroke. The ability of CNSS, e.g., MAP, to induce an extremely large release of these neurotransmitters in a very short time span may partially explain the neuroprotective effect we observed in our experiments. Future research aimed at understanding the neuroprotective mechanism of CNSS agents may further elucidate the exact mechanism and treatment for acute ischemic events.

### Example 4

### MCA Embolic Stroke Model in Adult Rats—1

### 4.1 Materials and Methods

Male Wistar rats at ages of 8-12 weeks, weighing 300-450 g were used in all experiments. A donor rat was anesthetized with 3.5% Isoflurane, and anesthesia was maintained with 1.0-1.5% Isoflurane in 70% N2O and 30% O2 using a face mask. Femoral arterial blood was withdrawn into lm of PE-50 tubing and retained in the tubing for 2 hours at room temperature, and subsequently retained for 22 h at 4° C. Four cm of the PE-50 tube containing rat clot was washed with saline for 5 minutes. A single rat clot (˜1 μl), was transferred to a modified PE-50 catheter with a 0.3 mm outer diameter filled with saline. Rats were then anesthetized with 3.5% Isoflurane, and anesthesia was maintained with 1.0-1.5% Isoflurane in 70% N2O and 30% O2 using a face mask throughout the surgical procedure. The animal's muzzle was placed in the face mask 2 cm from the surgical site. Rectal temperature was maintained at 37±″0.5° C. throughout the surgical procedure using an electric heating system. Under a surgical operating microscope) the right common carotid arteries (CCA), the right external carotid artery (ECA) and the internal carotid artery (ICA) were isolated via a 3 cm ventral neck midline incision. A 6-0 silk suture was loosely tied at the origin of the ECA and ligated at the distal end of the ECA. The right CCA and ICA was temporarily clamped using a curved microvascular clip (Codman & Shurtleff, Inc., Randolf, MAP, USA). A modified PE-50 catheter filled with a single clot (˜1 μl), was attached to a 100-μl Hamilton syringe, and introduced into the ECA lumen through a small puncture. The suture around the origin of the ECA was tightened around the intraluminal catheter to prevent bleeding, and the microvascular clip was removed. The catheter was gently advanced from the ECA into the lumen of the ICA. The clot along with 5 μl of saline in the catheter was injected into the ICA over 10 seconds. The catheter was withdrawn from the right ECA immediately after injection. The right ECA was ligated. The duration of the entire surgical procedure was approximately 25 min.

### Intravenous Administration of Methamphetamine or Saline:

Implantation of osmotic pumps for the purpose of continuous IV infusion occurred at both 6 and 12 hours after delivery of the 4cm clot. Experimental control for the experiment was achieved by substituting methamphetamine for physiological saline. Briefly, at 6 or 12 hours post stroke animals were re-anesthetized using 1-3% isoflurane. After a state of anesthesia was achieved the right side groin area was shaved. After shaving, surgical tape was utilized to remove excess hair. The area was scrubbed with betadine and allowed to dry.

A small incision was made and the groin area was blunt dissected to expose the femoral vein. The femoral vein was separated with surgical tweezers and the distal end was permanently ligated using 6-0 silk thread. The proximal end was ligated and a 0.2 mm incision (approximate) was made in the femoral vein. A 2.5 cm length of polyvinyl tubing (OD 0.07mm) connected to a pre-loaded osmotic pump (Alzet Corp. model 2001D; 6.6 microliters per hour for 24 hrs) was inserted into the vein and gently pushed up towards midline of the body. The tubing was inserted until 0.5 cm was exposed from the vein. The tubing was tied around the vein in two locations using 6-0 silk spaced approximately 2 mm apart. A small pocket was blunt dissected along the groin/abdominal area. The osmotic pump was inserted into the area on the outer wall of the abdomen underneath the skin and sutured into the abdominal fascia using 4-0 synthetic suture. The incision was closed using 4-0 synthetic suture. At 48-72 hours after initial insertion the animal was anesthetized, the groin area was scrubbed with betadine, the incision was reopened, blunt dissected, and the pump exposed. The sutures holding the pump and tubing in place were cut, the pump removed, and the femoral vein was permanently ligated using 6-0 silk suture. The pump was discarded and the incision was closed using 4-0 synthetic suture. The animal was monitored twice a day for 5 days to ensure they did not tear out external sutures or show signs of wound infection.

### Neurological Functional Tests:

Neurological functional tests were performed at 1, and 7 days after stroke onset.

Modified Neurological Severity Score (mNSS):

mNSS is composite of the motor (muscle status, abnormal movement), sensory (visual, tactile and proprioceptive) and reflex tests. For example, one of the motor tests, raising the rat by the tail: Flexion of forelimb—1 point, Flexion of hindlimb—1 point, Head moved more than 10° to the vertical axis within 30 seconds—1 point (see Table, below).

TABLE 1
Modified Neurological Severity Scores (mNSS) in Example 4

| Motor Tests | Points |
|---|---|
| Raising rat by the tail | 3 |
| Flexion of forelimb | 1 |
| Flexion of hindlimb | 1 |
| Head moved more than 10° to the vertical axis | 1 |
| within 30 seconds | |
| Walking on the floor (normal = 0; maximum = 3) | 3 |
| Normal walk | 0 |
| Inability to walk straight | 1 |
| Circling toward the paretic side | 2 |
| Fall down to the paretic side | 3 |
| Sensory tests: | 2 |
| Placing test (visual and tactile test) | 1 |
| Proprioceptive test (deep sensation, pushing the paw against | 1 |
| the table edge to stimulate limb muscles) | |
| Balance beam tests (normal = 0; maximum = 6) | 6 |
| Balances with steady posture | 0 |
| Grasps side of beam | 1 |
| Hugs the beam and one limb falls down from beam | 2 |
| Two limbs fall down from the beam, or spins | 3 |
| on beam (>60 sec) | |
| Attempts to balance on the beam but falls off (>40 sec) | 4 |
| Attempts to balance on the beam but falls off (>20 sec) | 5 |
| Falls off—no attempt to balance or hang onto | 6 |
| the beam (<20 sec) | |
| Absence of reflexes or abnormal movements | 4 |
| Pinna reflex (a head shake when touching the auditory meatus) | 1 |
| Corneal reflex (an eye blink when lightly touching | 1 |
| the cornea with cotton) | |
| Startle reflex (a motor response to a brief noise from | 1 |
| snapping a clipboard paper) | |
| Seizures, myoclonus, myodystony | 1 |
| MAXIMUM POINTS | 18 |

One point is awarded for the inability to perform the tasks or for the lack of a tested reflex. 13-18 severe injury; 7-12 moderate injury; 1-6 mild injury.

### Tissue Processing:

Rats were sacrificed at 7 days after MCA occlusion. The animals were euthanized using 15-20% isoflurane and decapitated immediately. The brain was removed and immersed in ice cold saline and then sectioned in a rat brain matrix (Activational Systems Inc., Warren, Mich.), into 7 coronal slabs (labeled A to G from front to back) each measuring 2.0 mm in thickness. Slices were immediately placed in 2% TTC and incubated at 37 degrees centigrade for 15 minutes. At the end of the incubation slices were thoroughly washed with prewarmed PBS and pictures were taken using a Nikon camera. All infarcts were analyzed using Image Pro Plus software utilizing the IOD function to assess the area and intensity of TTC staining Three dimensional infarct area was then obtained by inserting IOD data into a computational spreadsheet that was developed by Dr. Michael Chopp at Henry Ford Medical Center.

### 4.2 Results

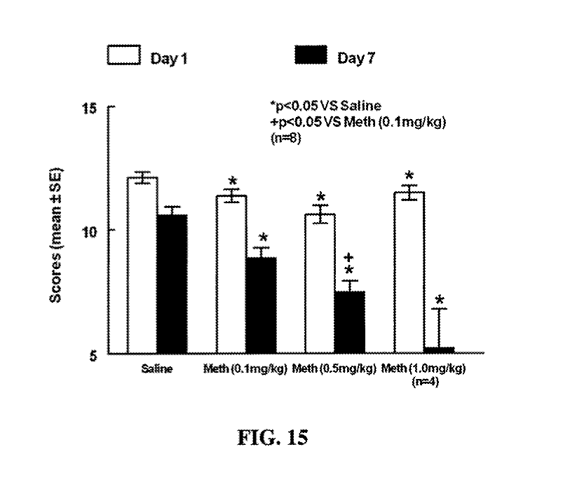

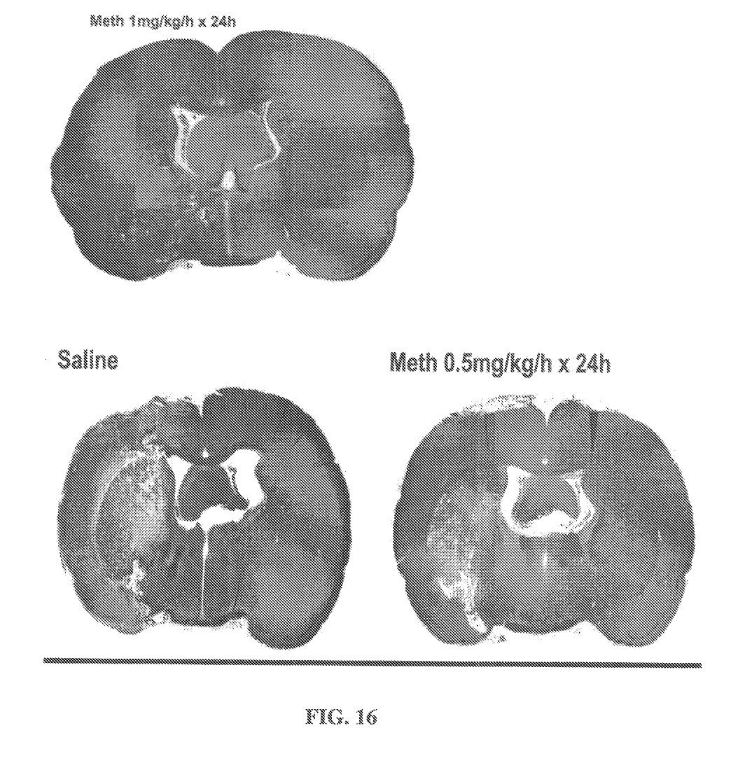

Initial experiments performed in the rat embolic model were done with intravenous infusion that began immediately after the clot was delivered and continued for 24 hours. Initial experiments demonstrated that a low dose of MAP (0.1 mg/kg/hr) failed to decrease the infarct size, but improved neurobehavioral outcomes. Increasing the dose to 0.5 and 1.0 mg/kg/hr decreased infarct size and improved neurobehavioral outcomes. Saline treated animals failed to show any significant improvement on any neurological outcome measure and showed infarcts that involved large areas of striatum and outer cortex. MAP treated animals at the two higher doses (0.5 and 1.0) showed a significant decrease in infracted area (FIG. 15).

FIG. 16 shows that methamphetamine administered at 0.5 and 1.0 mg/kg/hr immediately after embolic stroke reduces brain damage (infarct size) in adult rats. The infarct size were measured by TTC staining at 7 days post embolic stroke. Male Wistar rats were given a constant infusion of MAP (24 hrs) at 0.5 mg immediately after middle cerebral artery embolic occlusion. On day 7 coronal slices were made at 2.0 mm and stained with TTC. *=p<0.05; n=8

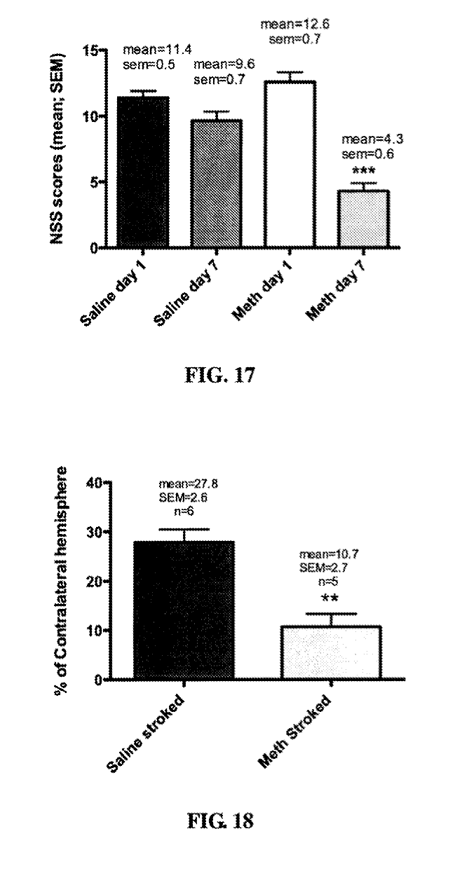

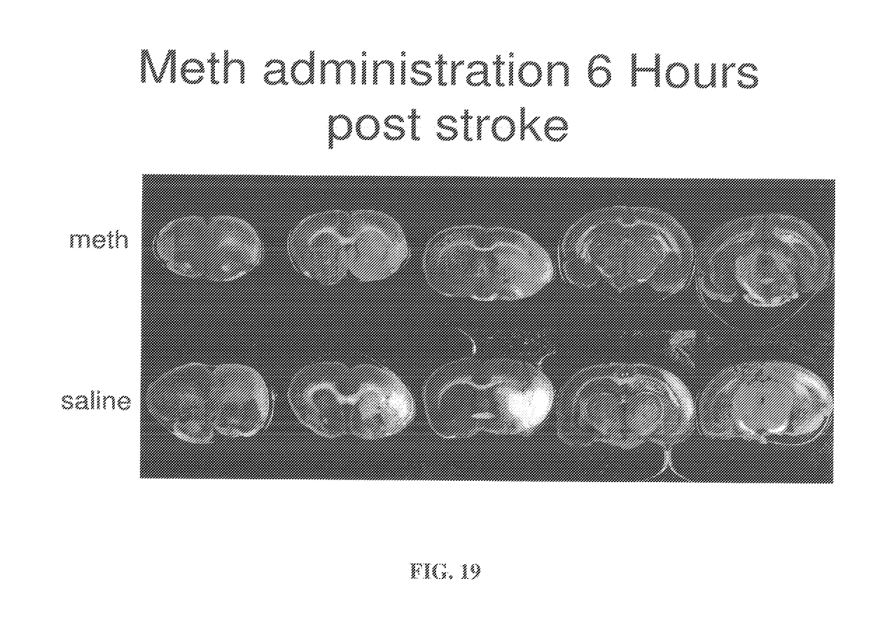

Of interest is the neurobehavioral improvement that occurred in the 0.1 mg/kg/hr group. This effect is unusual in that this improvement occurred without a significant reduction in infarct size. To further elucidate the effect of MAP after embolic stroke, animals were given an embolic stroke and then treated with a 1.0 mg/kg/hr dose that was started 6 hours after the clot was delivered. Animals were infused for 24 hours, the pump was removed and the animal was allowed to recover. Data collected from these experiments show that MAP delivered 6 hours after an embolic stroke significantly reduced infarct size and resulted in improved neurobehavioral outcomes on all testing parameters (FIGS. 17-19).

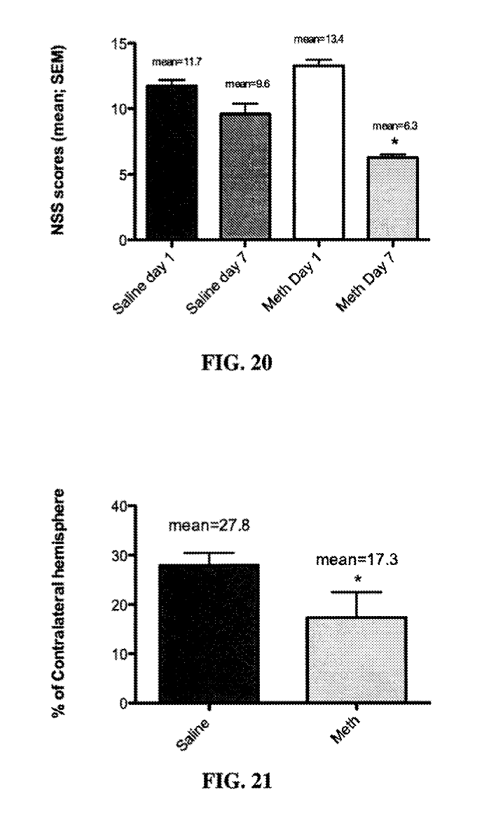

In light of the data collected at the 6 hour time point, we elected to perform a 12 hour delayed infusion in which the animals would receive MAP treatment 12 hours after the clot was delivered. Data collected from these experiments indicate MAP retains a robust effect on neurobehavioral outcomes, but shows a diminished effect on infarct size. While treatment at 12 hours still significantly reduces infarct size, the effect is significantly different from the 6 hour results (FIGS. 20-21).

### 4.3 Discussion

The data collected from these experiments indicate low dose MAP exerts a neuroprotective effect at both 6 and 12 hours after an embolic stroke. This observation represents a novel discovery in the field of stroke research. Until this point MAP has been viewed as a drug of abuse with limited potential for the clinical treatment of nervous system disorders.

### Example 5

### MCA Embolic Stroke Model in Adult Rats—2

### 5.1 Materials and Methods

### Animals

Thirty adult male Wistar rats were obtained from Charles River Labs and the

University of Montana Laboratory Animal Resources breeder colony. Animals were individually housed in a temperature and humidity controlled environment with 12 hr controlled light cycles. All animals were given free access to food and water. Following traumatic brain injury on Day 0, animals received AD Special veterinary diet to help maintain weight. All animals received 10 ml of saline and 10 ml of 5% dextrose daily via subcutaneous injection for 5 days (Days 0-5) to maintain hydration. The rats were 10-16 weeks old and weighed 350-500 grams at the initiation of test article administration (Day 0).

### Materials and Equipment

(+)Methamphetamine was obtained from SIGMA Chemical (St Louis, Mo.; Catalog No. M 8750; Lot No. 036K1052). A copy of the certificate of analysis for this lot is provided in Appendix 2. A stock solution of 100 mg/mL MA was prepared in sterile water and stored at 40 C for up to 2 weeks. The stock solution was diluted to 45.8-65.0 mg/mL in physiological saline (0.9% sodium chloride for injection) for administration. The dose concentration varied between animals, and was based on individual animal body weight on Day 0 and the dose concentration that would yield 0.5 mg/kg/hr MA at an infusion rate of 0.0073 mL/hr. The diluted solution was immediately loaded into an Alzet pump, which was then incubated at 370 C for 2 hrs prior to insertion into the animal.

The 0.9% sodium chloride was obtained from Baxter Healthcare Corp. (Deerfield, Ill.). Sterile 5% dextrose solution was obtained from Baxter Healthcare Corp. (Deerfield, Ill.). Alzet mini-osmotic pumps (Alzet; model 2001-D) were used to administer the test articles. PE-50 tubing was purchased from Scientific Commodities (Lake Havasu, Ariz.). Polyureathane tubing was obtained from (Scientific Commodities, Lake Havasu, Ariz.).

### Experimental Procedures

Three groups of male Wistar rats/group were used in this study. The animals were given 0 (vehicle control; saline), 0 (sham controls) or 0.5 mg/kg/hr MA via a 24 hr continuous IV infusion starting 3 hr after embolic stroke on Day 0. The test article was administered via an Alzet mini-osmotic pump at a rate of 0.0073 mL/hour. The dose concentration of MA loaded in the Alzet pump varied between animals (45.8-65.0 mg/mL), and was based on individual animal body weight on Day 0 and the dose concentration that would yield 0.5 mg/kg/hr MA at an infusion rate of 0.0073 mL/hr. Dosing of the animals was staggered over a 4-month period.

Prior to test article administration, all rats were subjected to a surgical procedure to produce a fluid percussion injury. Adult Sprague-Dawley rats (male, 350-500 g) are anesthetized with isoflurane anesthesia (1% isofluorane in 1 L/min O2) via face mask to effect for a small craniectomy (4.80 mm) and placement of a rigid Luer-loc needle hub (3 mm inside diameter). After luer lock hub placement, the hub is connected to the Cell Injury Controller through high pressure rubber tubing that delivers an fluid pulse to the dura. The fluid pressure pulse depresses the cortex and hippocampus producing an injury that closely mimicks a closed head trauma. A severe insult (2.00 ATM) was delivered and the rats were allowed to recover on a heating pad. The righting reflex time after injury was recorded. Animals were reanesthetized with isoflurane, and the luer hub was removed from the skull and the incision sutured using 4-0 absorbable sutures. An Alzet osmotic pump connected to a catheter was inserted into the femoral vein and sutured into the inguinal/flank area. A 200 uL bolus dose of 0.845 mg/kg/hr MA was delivered using a 30 gauge needle via the tail vein. This was followed by IV infusion of 0.5 mg/kg/hr of MA at a rate of 7.3 μl/hr for 24 hours. Animals were monitored 3-4 times a day for the first 48 hr following surgery and daily thereafter for 14 days.

After 24 hr of continuous infusion, the animals were anesthetized by isofluorane inhalation and the groin incision reopened. The pump and venous catheter were withdrawn and the right femoral vein was ligated. The incision was closed with 4-0 absorbable suture.

Behavioral testing was conducted at day 1. Efficacy parameters included NSS, and foot fault test at 30 hr, 7 days, 14 days, 21 days and 30 days after brain injury. Learning and memory (Morris Water Maze) was conducted on days 24-30. Neurological motor function was scored in all animals as follows:

Neurological severity score (NSS) was determined based on the scale below. One point was awarded for the inability to perform the task or for the lack of a tested reflex. In the case of walking on the floor and beam balance test, additional points were awarded as the severity of the finding increases. A maximum of 17 pts. was possible. \

TABLE 2
Neurological Severity Scores (NSS) in Example 5.

| Motor Tests | Points |
|---|---|
| Raising rat by the tail | 3 |
| Flexion of forelimb | 1 |
| Flexion of hindlimb | 1 |
| Head moved more than 10° to the vertical axis | 1 |
| within 30 seconds | |
| Walking on the floor (normal = 0; maximum = 3) | 3 |
| Normal walk | 0 |
| Inability to walk straight | 1 |
| Circling toward the paretic side | 2 |
| Fall down to the paretic side | 3 |
| Sensory tests: | 1 |
| Placing test (visual and tactile test) | 1 |
| Balance beam tests (normal = 0; maximum = 6) | 6 |
| Balances with steady posture | 0 |
| Grasps side of beam | 1 |
| Hugs the beam and one limb falls down from beam | 2 |
| Two limbs fall down from the beam, or spins | 3 |
| on beam (>60 sec) | |
| Attempts to balance on the beam but falls off (>40 sec) | 4 |
| Attempts to balance on the beam but falls off (>20 sec) | 5 |
| Falls off—no attempt to balance or hang onto | 6 |
| the beam (<20 sec) | |
| Absence of reflexes or abnormal movements | 4 |
| Pinna reflex (a head shake when touching the auditory meatus) | 1 |
| No vocalization when grasped behind the neck | 1 |
| Circle exit | 1 |
| Seeking behavior | 1 |
| MAXIMUM POINTS | 17 |

Foot fault testing was performed to measure placement dysfunction of forelimbs. Rats were placed on an elevated hexagonal wire grid. The rats must place their paws on the wire while moving along the grid. With each weight-bearing step, the paw may fall or slip between the wire. This was recorded as a foot fault. The total number of steps (movement of each forelimb) that the rat used to cross the grid were counted, and the total number of foot faults for each forelimb were recorded.

Morris water maze was used to assess the impact of MA on cognitive function (learning) following TBI. The maze consists of a large circular stock tank (7 ft diameter) painted black on the inside. The tank was filled to a depth of 1 ft and maintained at 25-28° C. with a heater. Several visual cues were placed on the walls around the water maze. These spatial cues remained constant throughout the experiment. A plexiglass escape platform (15 cm diameter) was placed in one of the quadrants of the maze. Rats completed four trials per day for 5 days. Each trial lasted a maximum of 120 sec. Each day the rat was placed in the water maze from one of the four start locations (i.e., north-east, south, east, south-west). Different start positions were chosen by the investigator performing the task for each animal on each day.

The rats were allowed to swim freely when placed in the water and given a maximum of 120 sec to find the submerged escape platform (2 cm below the water surface). The location of the hidden platform remained constant throughout the experiment. The latency or time to find the platform, swim speed, time spent in target quadrant and distance of swim path were recorded. All animals were allowed to remain on the platform for 15 sec to reinforce learning. Animals that fail to locate the platform in 120 sec were manually led to the platform and placed on it for 15 sec. After the completion of all four trials, animals were dried and warmed and returned to their home cages.

After all behavioral tests have been completed, rats in each experimental group were anaesthetized using 5% isofluorane collected, and then euthanatized via trans-cardiac perfusion with 4% paraformaldehyde in 0.1 M phosphate buffered saline (PBS), and brains harvested to evaluate infarct and collect tissue for further experiments. All data were collected by blinded observers and analyzed utilizing Prizm software. To determine Gaussian (normal) distribution a Kolmogorof-Smirnov test was performed on all data. Appropriate parametric analysis was performed on data sets containing two groups using an unpaired, two-tailed T-test (CI=95%). Analysis on three or more data sets was done using One-way ANOVA with Tukey's post-hoc to determine statistical significance between groups. A p<0.05 or less was considered significant.

### 5.2 Results

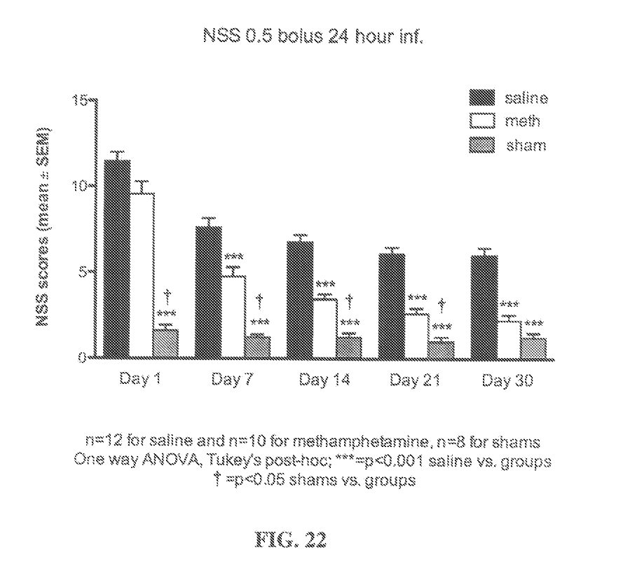

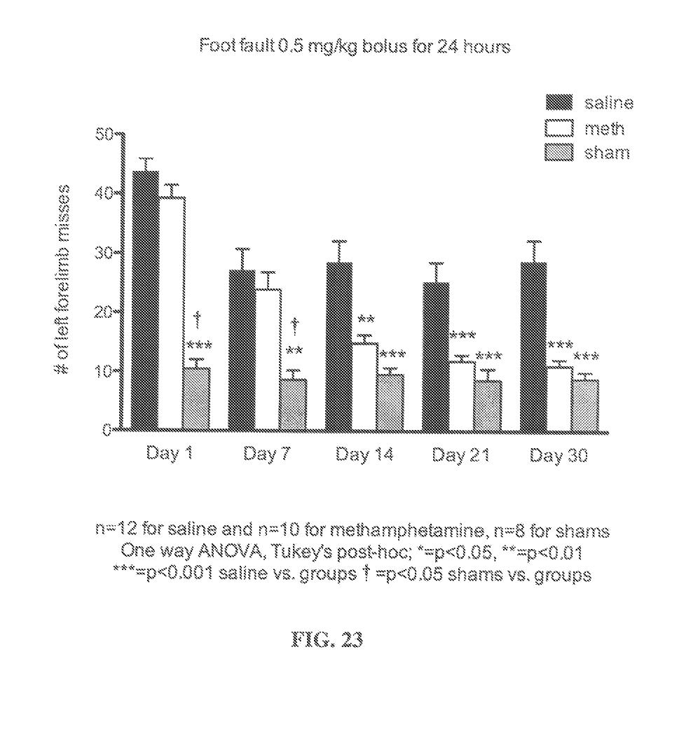

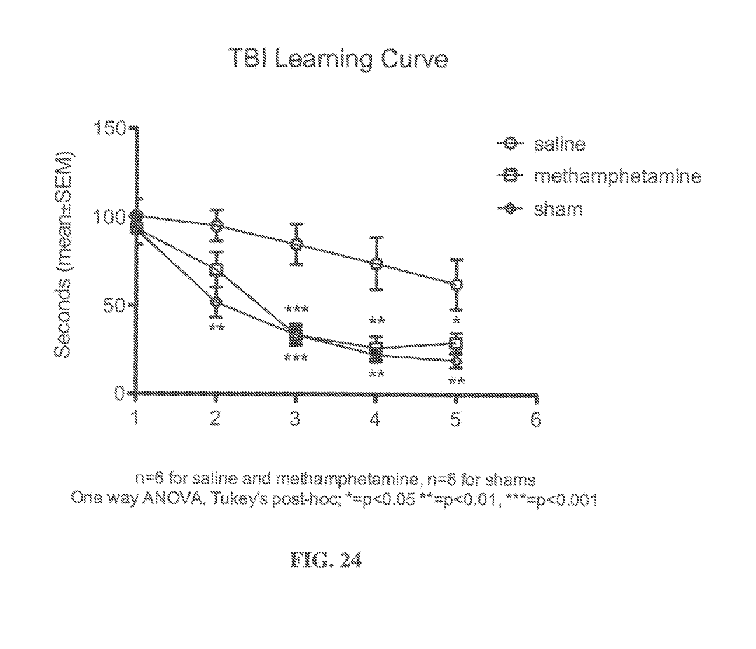

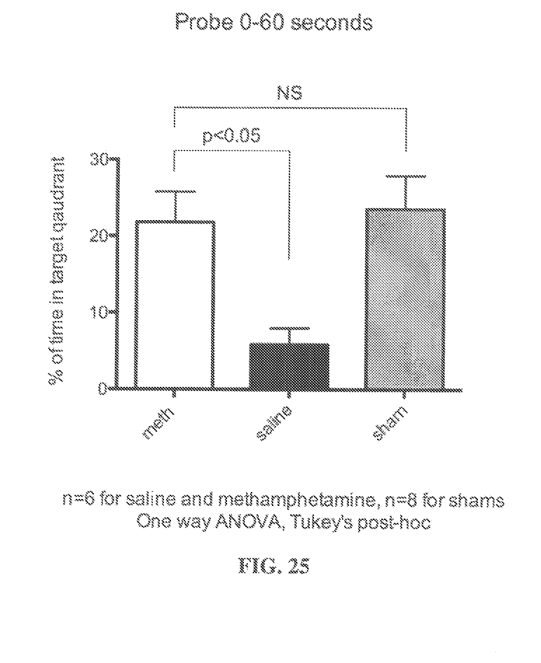

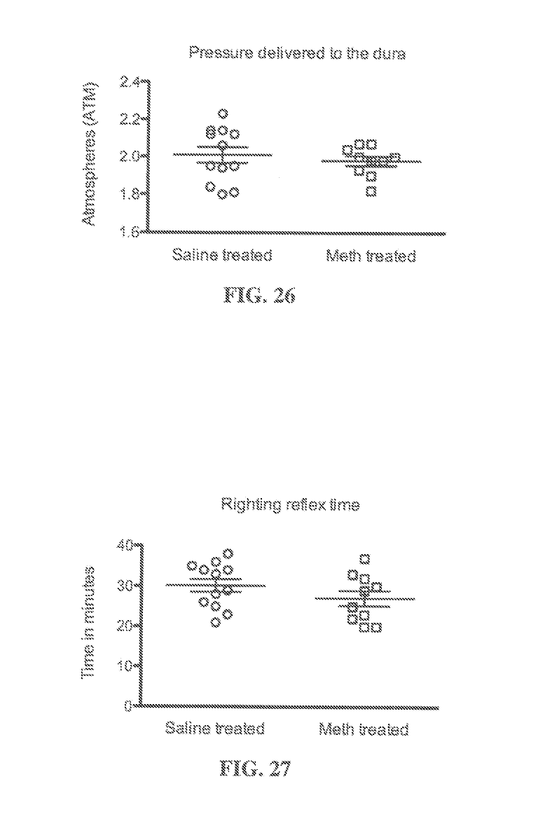

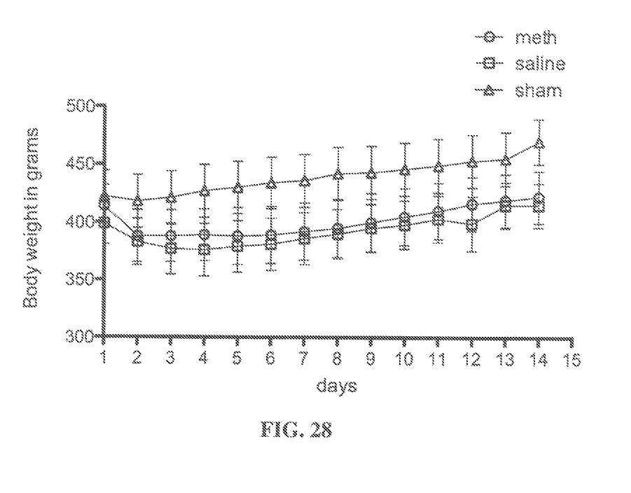

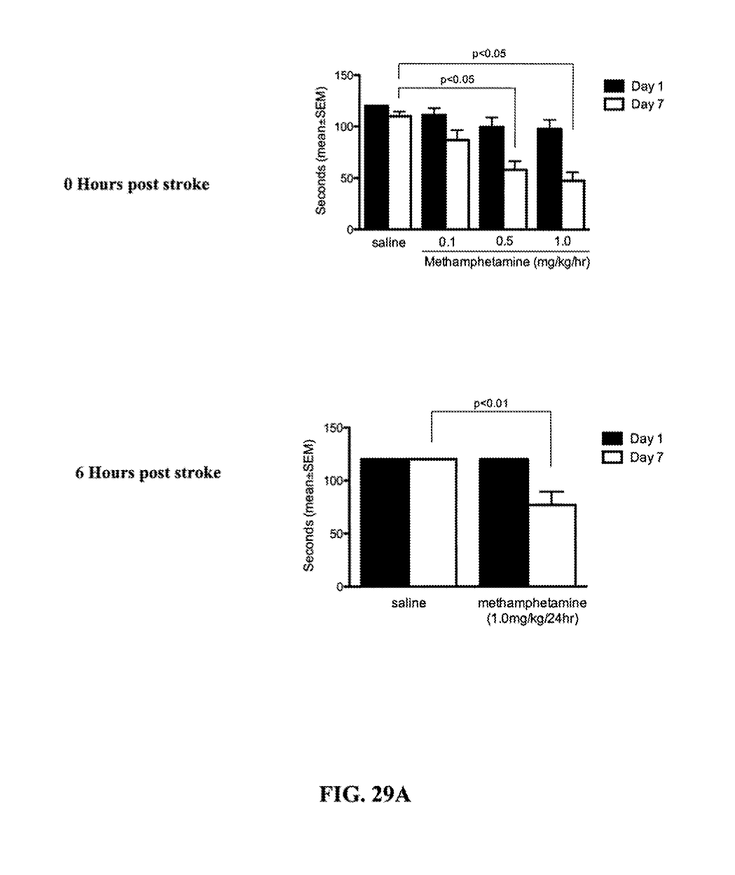

Results are presented in FIGS. 22-28. Significant differences in neurological severity scores were observed between the saline and MA treated groups when animals were assessed 7, 14, 21, and 30 days after traumatic brain injury (FIG. 22). At 7 days after stroke, statistically significant improvements in neurological severity scores (52% decrease with methamphetamine vs. 23% decrease with saline; p<0.01) were observed in the methamphetamine-treated animals compared to the saline controls. The methamphetamine was well tolerated with no toxicity and only changes due to the pharmacology of the drug.

### Example 6

### MCA Embolic Stroke Model in Adult Rats—3

### 6.1 Materials and Methods

Animal procedures were approved by the University of Montana and Henry Ford Hospital Institutional Animal Care and Use Committees. Focal embolic stroke was established as described in Example 4. Assessments of infarct volumes, modified neurological severity score, foot fault and adhesive tape removal were performed on days 1 and 7 as previously described in Example 4.

### 6.2 Results

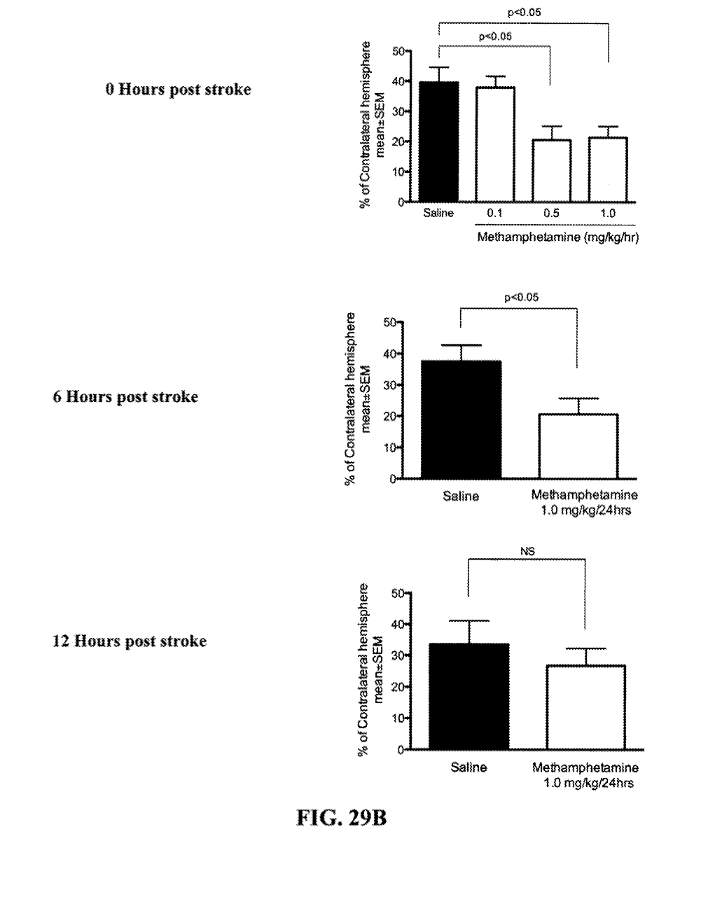

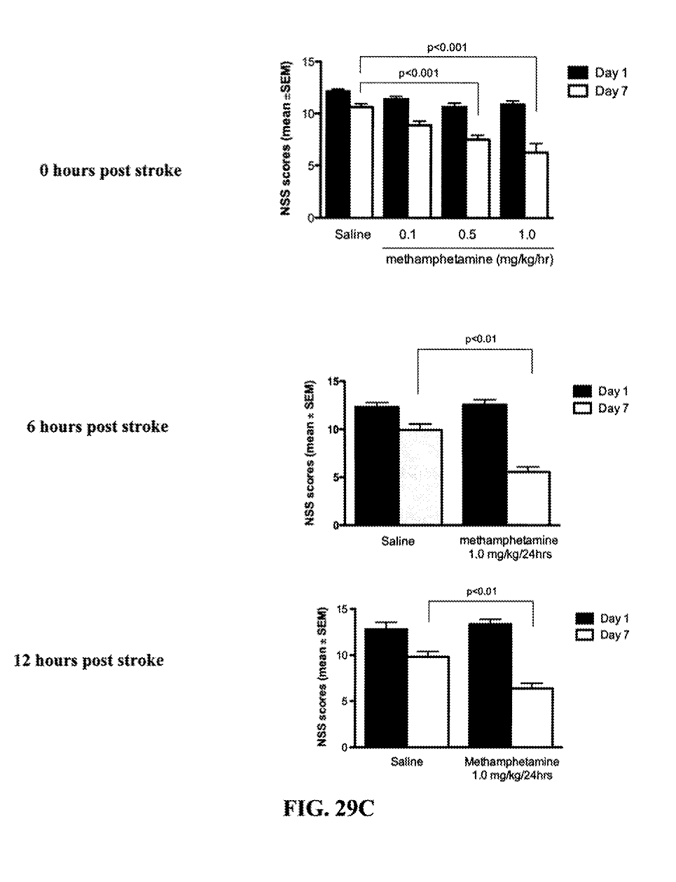

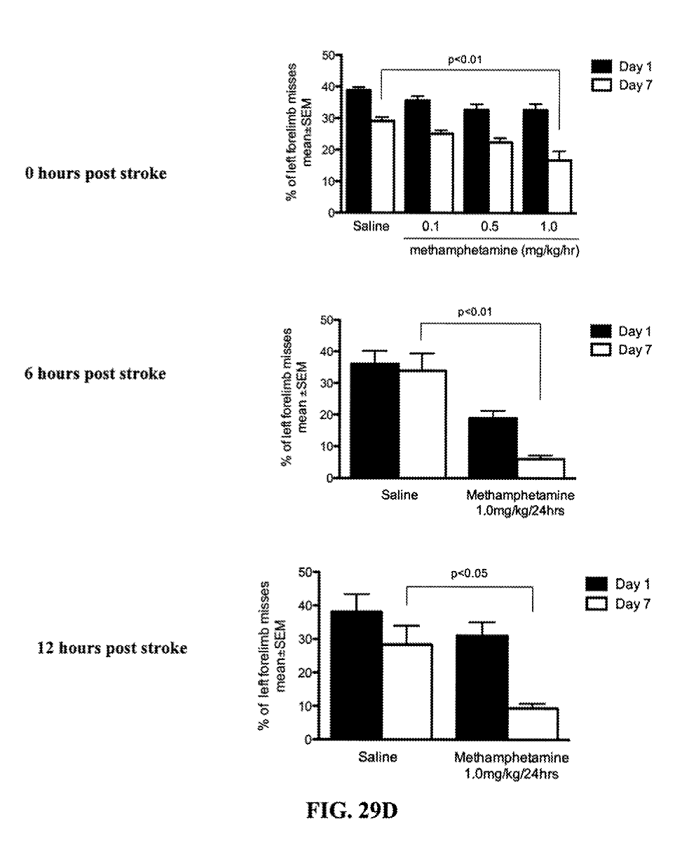

Focal ischemia was induced by the placement of a four cm fibrin clot within the right middle cerebral artery of adult male Wistar rats. Therapeutic efficacy was determined based on the assessment of: 1) infarct volumes, 2) neurological severity scores (NSS), 3) foot fault, and 4) adhesive tape removal. A dose dependent effect was observed when animals were administered 0.1, 0.5, or 1.0 mg/kg/hr through continuous IV infusion for 24 hrs beginning immediately after stroke (FIG. 29). There were no significant differences in the times required for animals to remove adhesive tape from their fore paws when tested 24 hours after stroke (FIG. 29a), indicating that all animals experienced strokes of similar severity. Likewise, there was no significant change in the time to remove tape between day 1 and day 7 for animals treated with either saline or 0.1 mg/kg/hr methamphetamine. In contrast, animals that received 0.5 or 1.0 mg/kg/hr methamphetamine immediately after stroke required significantly less time to remove the tape on day 7. This improvement in function was still observed when the 1 mg/kg/hr dose administration was started 6 hours after stroke (FIG. 29a). However, this same dose failed to produce an effect when administration was withheld until 12 hours after stroke (data not shown).

A similar dose response was observed for the improvement of infarct volumes when methamphetamine was delivered immediately after stroke (FIG. 29b). No significant reductions in infarct volumes were observed in animals treated with either saline or 0.1 mg/kg/hr methamphetamine. However, the two higher doses (0.5 and 1.0 mg/kg/hr) both provided significant reduction in infarct volumes. Furthermore, a significant reduction in infarct volumes was also observed with the 1.0 mg/kg/hr dose when treatment started 6 hours after stroke. Four out of the nine animals that received methamphetamine infusion beginning at 12 hours after stroke showed a substantial reduction in infarct volumes. However, when taken as a group the overall reduction in infarct volumes was not significantly different from the saline control group when treatment was initiated 12 hours after stroke.

As with infarct volumes, a significant improvement in neurological severity scores was observed for animals treated with 0.5 and 1.0 mg/kg/hr methamphetamine immediately after stroke or with 1.0 mg/kg/hr starting 6 hours after injury (FIG. 29c). However, in contrast to infarct volumes, a significant improvement in neurological severity scores was still observed in animals treated with 1.0 mg/kg/hr even when treatment was delayed until 12 hours after stroke. Similarly, significant improvements in foot fault values were observed at all three time points tested in animals treated with 1.0 mg/kg/hr methamphetamine (FIG. 29d).

### REFERENCES

Araki, H., Yamamoto, T., Kobayashi, Y., Futagami, K., Kawasaki, H., Gomita, Y. 2002. Effect of methamphetamine and imipramine on cerebral ischemia-induced hyperactivity in Mongolian gerbils. Japan Journal of Pharmacology 88, 293-299.

Arden, K. C., 2004. FoxO: linking new signaling pathways. Mol Cell 14, 416-418.

Babcock, A. M., Baker, D. A., Lovac, R. 1993. Locomotor activity in the ischemic gerbil. Brain Research 625, 351-354.

Behr, J., Gloveli, T., Schmitz, D., Heinemann, U., 2000. Dopamine depresses excitatory synaptic transmission onto rat subicular neurons via presynaptic D1-like dopamine receptors. J Neurophysiol 84, 112-119.

Boyeson, M. G., Feeney, D. M. 1990. Intraventricular norepinephrine facilitates motor recovery following sensorimotor cortex injury. Pharmacology Biochemistry Behavior 35, 497-501.

Boyeson, M. G., Harmon, R. L., Jones, J. L. 1994. Comparative effects of fluoxetine, amitriptyline and serotonin on functional motor recovery after sensorimotor cortex injury. American Journal Physical Medicine Rehabilitation 73, 76-83.

Brunet A., Bonni A., Zigmond M. J., Lin M. Z., Juo P., Hu L. S., Anderson M. J., Arden K. C., Blenis J., Greenberg M. E. Akt promotes cell survival by phosphorylating and inhibiting a forkhead transcription factor. Cell. 1999; 96:857-868.

Brunet A., Datta S. R., Greenberg M. E. Transcription-dependent and -independent control of neuronal survival by the pi3k-akt signaling pathway. Curr Opin Neurobiol. 2001; 11:297-305

Cadet J. L., Krasnova I. N., Jayanthi S., Lyles J. Neurotoxicity of substituted amphetamines: Molecular and cellular mechanisms. Neurotox Res. 2007; 11:183-202.

Cardozo-Pelaez, F., Cox, D. P., Bolin, C., 2005. Lack of the DNA repair enzyme OGG1 sensitizes dopamine neurons to manganese toxicity during development. Gene Expr 12, 315-323.

Cardozo-Pelaez, F., Song, S., Parthasarathy, A., Hazzi, C., Naidu, K., Sanchez-Ramos, J., 1999. Oxidative DNA damage in the aging mouse brain. Mov Disord 14, 972-980.

Chehab, N. H., Malikzay, A., Stavridi, E. S., Halazonetis, T. D., 1999. Phosphorylation of Ser-20 mediates stabilization of human p53 in response to DNA damage. Proc Natl Acad Sci U S A 96, 13777-13782.

Culmsee C., Semkova I., Krieglstein J. 1999. NGF mediates the neuroprotective effect of the beta₂-adrenoceptor agonist clenbuterol in vitro and in vivo: evidence from an NGF-antisense study. Neurochemistry International 35, 47-57.

Datta S.R., Dudek H., Tao X., Masters S., Fu H., Gotoh Y., Greenberg ME. Akt phosphorylation of bad couples survival signals to the cell-intrinsic death machinery. Cell. 1997; 91:231-241.

Dhanasekaran A., Gruenloh S. K., Buonaccorsi J. N., Zhang R., Gross G. J., Falck J. R., Patel P. K., Jacobs E. R., Medhora M. Multiple antiapoptotic targets of the pi3k/akt survival pathway are activated by epoxyeicosatrienoic acids to protect cardiomyocytes from hypoxia/anoxia. Am J Physiol Heart Circ Physiol. 2008; 294:H724-735.

Dietrich W. D., Alonso O., Busto R., Watson B. D., Loor Y., Ginsberg M. D. 1990. Influence of amphetamine treatment on somatosensory function of the normal and infarcted rat brain. Stroke 21, 111147-111150.

Dudek H., Datta S. R., Franke T. F., Birnbaum M. J., Yao R., Cooper G. M., Segal R. A., Kaplan D. R., Greenberg M. E. Regulation of neuronal survival by the serine-threonine protein kinase akt. Science. 1997; 275:661-665.

Feeney D. M., Gonzalez A., Law W. A. 1982. Amphetamine, haloperidol, and experience interact to affect rate of recovery after motor cortex injury. Science 217, 855-857.

Fiskum, G., Rosenthal, R. E., Vereczki, V., Martin, E., Hoffman, G. E., Chinopoulos, C., Kowaltowski, A., 2004. Protection against ischemic brain injury by inhibition of mitochondrial oxidative stress. J Bioenerg Biomembr 36, 347-352.

Follesa P., Mocchetti I. 1993. Regulation of basic fibroblast growth factor and nerve growth factor mRNA by beta-adrenergic receptor activation and adrenal steroids in rat central nervous system. Molecular Pharmacology 43, 132-138.

Gold P. E., Delanoy R. L., Merrin J. 1984. Modulation of long-term potentiation by peripherally administered amphetamine and epinephrine. Brain Research 305, 103-107.

Hanson, G. R., Gibb, J. W., Metzger, R. R., Kokoshka, J. M., Fleckenstein, A. E., 1998. Methamphetamine-induced rapid and reversible reduction in the activities of tryptophan hydroxylase and dopamine transporters: oxidative consequences? Ann N Y Acad Sci 844, 103-107.

Honda, R., Tanaka, H., Yasuda, H., 1997. Oncoprotein MDM2 is a ubiquitin ligase E3 for tumor suppressor p53. FEBS Lett 420, 25-27.

Hovda D. A., Feeney D. M. 1985. Haloperidol blocks amphetamine induced recovery of binocular depth perception after bilateral visual cortex ablation in cat. Proceedings Western Pharmacology Society 28, 209-211.

Hovda D. A., Fenney D. M. 1984. Amphetamine with experience promotes recovery of locomotor function after unilateral frontal cortex injury in the cat. Brain Research 298, 358-361.

Hovda D. A., Sutton R. L., Feeney D. M. 1987. Recovery of tactile placing after visual cortex ablation in cat: a behavioral and metabolic study of diaschisis. Experimental Neurology 97, 391-402.

Jeong, S. J., Dasgupta, A., Jung, K. J., Um, J. H., Burke, A., Park, H. U., Brady, J. N., 2008. PI3K/AKT inhibition induces caspase-dependent apoptosis in HTLV-1-transformed cells. Virology 370, 264-272.

Katz, L. M., Lotocki, G., Wang, Y., Kraydieh, S., Dietrich, W. D., Keane, R. W., 2001. Regulation of caspases and XIAP in the brain after asphyxial cardiac arrest in rats. Neuroreport 12, 3751-3754.

Kiang, J. G., Tsen, K. T., 2006. Biology of hypoxia. Chin J Physiol 49, 223-233.

Kihara, T., Shimohama, S., Sawada, H., Honda, K., Nakamizo, T., Kanki, R., Yamashita, H., Akaike, A., 2002. Protective effect of dopamine D2 agonists in cortical neurons via the phosphatidylinositol 3 kinase cascade. J Neurosci Res 70, 274-282.

Kirino T. 1982. Delayed neuronal death in the gerbil hippocampus following ischemia. Brain Research 239, 57-69.

Kirino T., Sano K. Selective vulnerability in the gerbil hippocampus following transient ischemia. Acta Neuropathol. 1984; 62:201-208.

Kita T., Miyazaki I., Asanuma M., Takeshima M., Wagner G. C. Dopamine-induced behavioral changes and oxidative stress in methamphetamine-induced neurotoxicity. Int Rev Neurobiol. 2009; 88:43-64.

Koh, P. O., 2008. Melatonin attenuates the focal cerebral ischemic injury by inhibiting the dissociation of pBad from 14-3-3. J Pineal Res 44, 101-106.

Krasnova I. N., Cadet J. L. Methamphetamine toxicity and messengers of death. Brain Res Rev. 2009; 60:379-407.

Kwon, D. S., Kwon, C. H., Kim, J. H., Woo, J. S., Jung, J. S., Kim, Y. K., 2006. Signal transduction of MEK/ERK and PI3K/Akt activation by hypoxia/reoxygenation in renal epithelial cells. Eur J Cell Biol 85, 1189-1199.

Lee F. J., Xue S., Pei L., Vukusic B., Chery N., Wang Y., Wang Y. T., Niznik H. B., Yu X. M., Liu F. Dual regulation of nmda receptor functions by direct protein-protein interactions with the dopamine dl receptor. Cell. 2002; 111:219-230.

Leinninger, G. M., Backus, C., Uhler, M. D., Lentz, S. I., Feldman, E. L., 2004. Phosphatidylinositol 3-kinase and Akt effectors mediate insulin-like growth factor-I neuroprotection in dorsal root ganglia neurons. FASEB J 18, 1544-1546.

Liu, X. Y., Chu, X. P., Mao, L. M., Wang, M., Lan, H. X., Li, M. H., Zhang, G. C., Parelkar, N. K., Fibuch, E. E., Haines, M., Neve, K. A., Liu, F., Xiong, Z. G., Wang, J. Q., 2006. Modulation of D2R-NR2B interactions in response to cocaine. Neuron 52, 897-909.

Martinsson L., Eksborg S. 2004. Drugs for stroke recovery: the example of amphetamines. Drugs Aging 21, 67-79.

Meley D., Pattingre S., Codogno P. [pi3 kinases and the control of autophagia]. Bull Cancer. 2006; 93:439-444.

Mintz M., Tomer R. 1986. Exposure to amphetamine after substantia nigra lesion interferes with the process of behavioral recovery. Pharmacology Biochemistry Behavior 25, 1307-1311.

Miyawaki, T., Mashiko, T., Ofengeim, D., Flannery, R. J., Noh, K. M., Fujisawa, S., Bonanni, L., Bennett, M. V., Zukin, R. S., Jonas, E. A., 2008. Ischemic preconditioning blocks BAD translocation, Bc1-xL cleavage, and large channel activity in mitochondria of postischemic hippocampal neurons. Proc Natl Acad Sci U S A 105, 4892-4897.

Nash, J. F., Yamamoto, B. K., 1993. Effect of D-amphetamine on the extracellular concentrations of glutamate and dopamine in iprindole-treated rats. Brain Res 627, 1-8.

Noraberg, J., Kristensen, B. W., Zimmer, J., 1999. Markers for neuronal degeneration in organotypic slice cultures. Brain Res Brain Res Protoc 3, 278-290.

O'Phelan K., McArthur D. L., Chang C. W., Green D., Hovda D. A. The impact of substance abuse on mortality in patients with severe traumatic brain injury. J Trauma. 2008; 65:674-677.

Plas D. R., Talapatra S., Edinger A. L., Rathmell J. C., Thompson C. B. Akt and bc1-xl promote growth factor-independent survival through distinct effects on mitochondrial physiology. J Biol Chem. 2001; 276:12041-12048.

Quinton, M. S., Yamamoto, B. K., 2006. Causes and consequences of methamphetamine and MDMA toxicity. Aaps J 8, E337-347.

Schmanke T. D., Avery R. A., Barth T. M. 1996. The effects of amphetamine on recovery of function after cortical damage in the rat depend on the behavioral requirements of the task. Journal of Neurotrauma 13, 293-307.

Selkirk, J. V., Nottebaum, L. M., Vana, A. M., Verge, G. M., Mackay, K. B., Stiefel, T. H., Naeve, G. S., Pomeroy, J. E., Petroski, R. E., Moyer, J., Dunlop, J., Foster, A. C., 2005. Role of the GLT-1 subtype of glutamate transporter in glutamate homeostasis: the GLT-1-preferring inhibitor WAY-855 produces marginal neurotoxicity in the rat hippocampus. Eur J Neurosci 21, 3217-3228.

Semkova I., Schilling M., Henrich-Noack P., Rami A., Krieglstein J. 1996. Clenbuterol protects mouse cerebral cortex and rat hippocampus from ischemic damage and attenuates glutamate neurotoxicity in cultured hippocampal neurons by induction of NGF. Brain Research 717, 44-54.

Smith, K. J., Self, R. L., Butler, T. R., Mullins, M. M., Ghayoumi, L., Holley, R. C., Littleton, J. M., Prendergast, M. A., 2007. Methamphetamine exposure antagonizes N-methyl-D-aspartate receptor-mediated neurotoxicity in organotypic hippocampal slice cultures. Brain Res 1157, 74-80.

Squire L.R., Zola-Morgan S. 1991. The medial temporal lobe memory system. Science 253, 1380-1386.

Stroemer R. P., Kent T. A., Hulsebosch C. E. 1998. Enhanced neocortical neural sprouting, synaptogenesis, and behavioral recovery with D-amphetamine therapy after neocortical infarction in rats. Stroke 29, 2381-2393.

Sutton R. L., Hovda D. A., Chen M. J., Feeney D. M. 2000. Alleviation of brain injury-induced cerebral metabolic depression by amphetamine, a cytochrome oxidase histochemistry study. Neural Plasticity 7, 109-125.

Sutton R. L., Hovda D. A., Feeney D. M. 1989. Amphetamine accelerates recovery of locomotor function following bilateral frontal cortex ablation in cats. Behavioral Neuroscience 103, 837-841.

Teuchert-Noodt G, Dawirs R R, Hildebrandt K. 2000. Adult treatment with methamphetamine transiently decreases dentate granule cell proliferation in the gerbil hippocampus. Journal Neural Transmission 107, 133-143.

Thrash B., Thiruchelvan K., Ahuja M., Suppiramaniam V., Dhanasekaran M. Methamphetamine-induced neurotoxicity: The road to parkinson's disease. Pharmacol Rep. 2009; 61:966-977.

Uchiyama T., Engelman R. M., Maulik N., Das D. K. Role of akt signaling in mitochondrial survival pathway triggered by hypoxic preconditioning. Circulation. 2004; 109:3042-3049.

Wang D., Corbett D. 1990. Cerebral ischemia, locomotor activity and spatial mapping. Brain Research 533, 78-82.

Wang, L. L., Ou, C. C., Chan, J. Y., 2005. Receptor-independent activation of GABAergic neurotransmission and receptor-dependent nontranscriptional activation of phosphatidylinositol 3-kinase/protein kinase Akt pathway in short-term cardiovascular actions of dexamethasone at the nucleus tractus solitarii of the rat. Mol Pharmacol 67, 489-498.

Wang, Q., Liu, L., Pei, L., Ju, W., Ahmadian, G., Lu, J., Wang, Y., Liu, F., Wang, Y. T., 2003. Control of synaptic strength, a novel function of Akt. Neuron 38, 915-928.

Wang Y., Hayashi T., Chang C. F., Chiang Y. H., Tsao L. I., Su T. P., Borlongan C., Lin S. Z. 2001. Methamphetamine potentiates ischemia/reperfusion insults after transient middle cerebral artery ligation. Stroke 32, 775-782.

Zhang R. L., Chopp M., Zhang Z. G., Jiang Q., Ewing J. R. A rat model of focal embolic cerebral ischemia. Brain Res. 1997; 766:83-92.

Zhang Z., Zhang R. L., Jiang Q., Raman S. B., Cantwell L., Chopp M. A new rat model of thrombotic focal cerebral ischemia. J Cereb Blood Flow Metab. 1997; 17:123-135.

Zola-Morgan S., Squire L. R., Amaral D. G. 1986. Human amnesia and the medial temporal region: Enduring memory impairment following a bilateral lesion limited to field CA1 of the hippocampus. Journal of Neuroscience 6, 2960-2967.

Zou S., Li L., Pei L., Vukusic B., Van Tol H. H., Lee F. J., Wan Q., Liu F. Protein-protein coupling/uncoupling enables dopamine d2 receptor regulation of ampa receptor-mediated excitotoxicity. J Neurosci. 2005; 25:4385-4395.

1. A method of reducing the occurrence of brain cell death caused by transient cerebral hypoxia, ischemia, or traumatic brain injury, the method comprising identifying a subject suffering from transient cerebral hypoxia, ischemia, or traumatic brain injury, and within 12 hours of the onset of the condition or injury, administering to the subject a continuous intravenous infusion dose of methamphetamine in an amount sufficient to reduce the occurrence of brain cell death caused by the transient cerebral hypoxia, ischemia, or traumatic brain injury.

2. The method of claim 1, wherein administration occurs within hours of the onset of the condition or injury and further comprises administering a bolus dose of methamphetamine to the subject of up to 0.18 mg/kg.

3. The method of claim 2, wherein the amount sufficient to reduce the occurrence of brain cell death increases in correspondence to an increase in the amount of time between the onset of the condition or injury and the initial administration of methamphetamine.

4. The method of claim 3, wherein the continuous infusion dose is initially administered within 6 hours after the onset of the transient cerebral hypoxia, ischemia, or traumatic brain injury and the continuous infusion dose is less than or equal to 0.5 mg/kg/hr.

5. The method of claim 2, wherein the continuous infusion dose is administered for at least 6 hours at up to 0.5 mg/kg/hr or less.

6. The method of claim 1, wherein the continuous intravenous infusion is about 0.07 mg/kg/hr or less and is in combination with a bolus dose.

7. (canceled)

8. The method of claim 1, wherein the amount administered is sufficient to increase the expression of IL-10 and/or decrease the expression of IL-6.

9-10. (canceled)

11. The method of claim 1, wherein the amount administered is sufficient to reduce apoptosis of brain cells caused by the condition.

12. The method of claim 1, wherein the subject is a human and the amount of methamphetamine administered is sufficient to obtain a steady state plasma concentration of about 0.01 mg/L to about 0.05 mg.

13. (canceled)

14. The method of claim 1, wherein the amount sufficient to modulate cytokine expression within the brain is less than or equal to 1.0 mg/kg/hr.

15. The method of claim 1, wherein the amount of methamphetamine administered is sufficient to increase the expression of Bc1-2 and Bc1-x_{L} in the subject.

16. The method of claim 1, wherein the amount of methamphetamine administered is sufficient to activate NF-kB and CREB in the subject.

17. The method of claim 1, wherein the neurotrophins are selected from the group consisting of BDNF, NT3, and NPY.

18. The method of claim 1, wherein the methamphetamine is administered within 6 hours after onset of the condition and is administered together over a 24 hour period at 40 mg or less.

19. The method of claim 1, wherein method reduces the occurrence of brain death in the hippocampus.

20. The method of claim 1, wherein the condition is caused by traumatic brain injury.

21. The method of claim 1, wherein the subject is a human.

22. The method of claim 1, wherein the methamphetamine is administered within 2 hours of surgery.

23. The method of claim 1, wherein the a continuous intravenous infusion consist of a therapeutically effective amount of (+)-methamphetamine and a pharmaceutically acceptable carrier.

24. The method of claim 1, wherein the amount of methamphetamine administered is sufficient to obtain a steady state plasma concentration of about 0.01 mg/L to about 0.3 mg/L in less than an hour.
